(19) United States
(12) Patent Application Publication
(10) Pub. No.: US 2023/0402176 A1
(43) Pub. Date: Dec. 14, 2023

(54) SYSTEMS AND METHODS FOR CONTROLLING MEDICAL OPERATIONS

(71) Applicant: SHANGHAI UNITED IMAGING HEALTHCARE CO., LTD., Shanghai (CN)

(72) Inventor: Ruirui JIANG, Shanghai (CN)

(73) Assignee: SHANGHAI UNITED IMAGING HEALTHCARE CO., LTD., Shanghai (CN)

(21) Appl. No.: 18/332,666

(22) Filed: Jun. 9, 2023

(30) Foreign Application Priority Data
Jun. 9, 2022 (CN) 202210647682.X
Jun. 22, 2022 (CN) 202210713199.7

(51) Int. Cl. G16H 40/67

(52) U.S. Cl. CPC G16H 40/67

(57) ABSTRACT

The embodiments of the present disclosure provide systems and methods for controlling medical operations. The system may include: at least one storage medium including a set of instructions; and at least one processor in communication with the at least one storage medium, wherein when executing the set of instructions, the at least one processor is directed to cause the system to perform operations including: obtaining a call instruction of virtual caregiver information, wherein the virtual caregiver information includes accompany information associated with a caregiver, the caregiver is related to an object, and the accompany information includes at least one of visual information, auditory information, or tactile information; and directing a display device to display the virtual caregiver information based on the call instruction.

### CROSS-REFERENCE TO RELATED APPLICATIONS

This application claims priority to Chinese Patent Application No. 202210647682.X, filed on Jun. 9, 2022, and Chinese Patent Application No. 202210713199.7, filed on Jun. 22, 2022, the entire contents of which are hereby incorporated by reference.

### TECHNICAL FIELD

The present disclosure generally relates to the field of medical technology, and in particular, to systems and methods for controlling medical operations.

### BACKGROUND

In recent years, medical imaging technology has been widely used in clinical examination and medical diagnosis. A medical device (e.g., a computed tomography (CT) scanning device, a positron emission computed tomography (PET) scanning device) can obtain pathological information of lesions in a patient through medical imaging. However, patients are often unaccompanied during medical scanning and is incapable of controlling the scanning process or providing feedback. Therefore, it is desirable to provide systems and methods for controlling medical operations.

### SUMMARY

In one aspect of the present disclosure, a system is provided. The system may include at least one storage medium including a set of instructions; and at least one processor in communication with the at least one storage medium, wherein when executing the set of instructions, the at least one processor is directed to cause the system to perform operations including: obtaining a call instruction of virtual caregiver information, wherein the virtual caregiver information includes accompany information associated with a caregiver, the caregiver is related to an object, and the accompany information includes at least one of visual information, auditory information, or tactile information; and directing a display device to display the virtual caregiver information based on the call instruction.

In another aspect of the present disclosure, a method is provided. The method may include obtaining a call instruction of virtual caregiver information, wherein the virtual caregiver information includes accompany information associated with a caregiver, the caregiver is related to an object, and the accompany information includes at least one of visual information, auditory information, or tactile information; and directing a display device to display the virtual caregiver information based on the call instruction.

In still another aspect of the present disclosure, a system is provided. The system may include at least one storage medium including a set of instructions; and at least one processor in communication with the at least one storage medium, wherein when executing the set of instructions, the at least one processor is directed to cause the system to perform operations including: obtaining twin data associated with an object from an operation terminal device; transmitting the twin data associated with the object to a control terminal device; obtaining guidance data from the control terminal device, wherein the guidance data is determined based on the twin data associated with the object; and directing a display device associated with the operation terminal device to present the guidance data.

Additional features will be set forth in part in the description which follows, and in part will become apparent to those skilled in the art upon examination of the following and the accompanying drawings or may be learned by production or operation of the examples. The features of The present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating exemplary hardware and/or software components of an exemplary computing device according to some embodiments of the present disclosure;

FIG. 3 is a block diagram illustrating an exemplary processing device according to some embodiments of the present disclosure;

FIG. 4 is a flowchart illustrating an exemplary process for controlling a medical operation according to some embodiments of the present disclosure;

FIG. 5 is a flowchart illustrating an exemplary process for determining an instruction with respect to reference information associated with a medical operation according to some embodiments of the present disclosure;

FIG. 6 is a flowchart illustrating an exemplary process for providing virtual caregiver information during a medical operation according to some embodiments of the present disclosure;

FIGS. 7A and 7B are schematic diagrams illustrating an exemplary process for providing virtual caregiver information during a medical operation according to some embodiments of the present disclosure;

FIG. 8 is a flowchart illustrating an exemplary process for adjusting an operational parameter of a medical operation according to some embodiments of the present disclosure;

FIG. 9 is a flowchart illustrating an exemplary process for adjusting an instruction with respect to reference information associated with a medical operation and/or the reference information according to some embodiments of the present disclosure;

FIG. 10 is a flowchart illustrating an exemplary process for providing virtual caregiver information during a medical operation according to some embodiments of the present disclosure;

FIG. 11 is a flowchart illustrating an exemplary process for remote medical operation according to some embodiments of the present disclosure;

FIG. 12 is a flowchart illustrating an exemplary process for remote medical operation according to other embodiments of the present disclosure;

FIG. 13 is a flowchart illustrating an exemplary process for remote medical operation according to other embodiments of the present disclosure;

FIG. 14 is a schematic diagram illustrating an exemplary scene of a first space and a second space according to some embodiments of the present disclosure; and

FIGS. 15A and 15B are schematic diagrams illustrating exemplary medical scenes according to some embodiments of the present disclosure.

### DETAILED DESCRIPTION

In order to more clearly illustrate the technical solutions associated with the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that the “system,” “device,” “unit,” and/or “module” used herein are one method to distinguish different components, elements, parts, sections, or assemblies of different levels. However, if other words can achieve the same purpose, the words can be replaced by other expressions.

As used in the disclosure and the appended claims, the singular forms “a,” “an,” and “the” include plural referents unless the content clearly dictates otherwise; the plural forms may be intended to include singular forms as well. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The terms “comprise,” “comprises,” “comprising,” “include,” “includes,” “including,” “have,” “has,” “having,” and any variations thereof referred to in the present disclosure are intended to cover non-exclusive inclusions. For example, a process, a method, a system, a product, or a device including a series of operations or modules (units) is not limited to the operations or units listed, but may also include operations or units that are not listed, or may also include other operations or units inherent to the process, the method, the product or the device. The “a plurality of” referred to in the present disclosure refers to greater than or equal to two. “And/or” describes an association relationship of associated objects, indicating that three kinds of relationships may exist, for example, “A and/or B” may indicate that A exists alone, A and B exist simultaneously, and B exists alone. The terms “first,” “second,” “third,” and “fourth,” etc. referred to in the present disclosure are only to distinguish similar objects, and do not represent a specific order for the objects.

The flowcharts used in the present disclosure illustrate operations that systems implement according to some embodiments in the present disclosure. Relevant descriptions is provided to assist in a better understanding of medical imaging methods and/or systems. It is to be expressly understood, the operations of the flowchart may be implemented not in order. Conversely, the operations may be implemented in inverted order, or simultaneously. Moreover, one or more other operations may be added to the flowcharts. One or more operations may be removed from the flowcharts.

In the present disclosure, the terms “object,” “scanning object,” “target object,” “target,” or “detected object” can be used interchangeably.

Generally, during a medical operation (e.g., a medical scanning process), a patient is often unaccompanied and only can communicate with a scanning technician through an audio device such as a microphone or a loudspeaker, which may result in an uncomfortable experience or result in patient being nervous or anxious. Accordingly, the embodiments of the present disclosure provide systems and methods for controlling a medical operation, thereby improving patient experience and improving medical imaging efficiency. The system may obtain a call instruction of virtual caregiver information, and direct a display device to display the virtual caregiver information based on the call instruction.

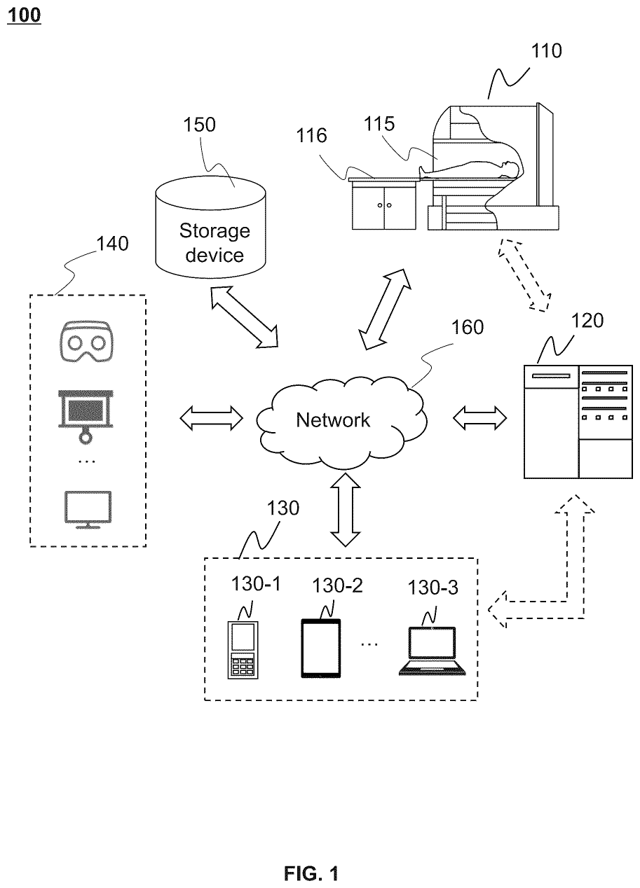

FIG. 1 is a schematic diagram illustrating an exemplary medical system according to some embodiments of the present disclosure. As shown in FIG. 1, a medical system 100 may include a medical device 110, a processing device 120, a terminal device 130, a display device 140, a storage device 150, and a network 160. The components in the medical system 100 may be connected to each other in various ways. Merely by way of example, as shown in FIG. 1, the medical device 110 may be connected to the processing device 120 via the network 160. As another example, the medical device 110 may be directly connected to the processing device 120 (as shown by a two-way arrow in a dotted line connecting the medical device 110 and the processing device 120). As still another example, the storage device 150 may be connected to the processing device 120 directly or via the network 160. As still another example, the display device 140 may be connected to the processing device 120 via the network 160. As a further example, the terminal device 130 may be directly connected to the processing device 120 (as indicated by a two-way arrow in a dotted line connecting the terminal device 130 and the processing device 120) or connected to the processing device 120 via the network 160.

The medical device 110 may scan an object and/or generate data associated with the object. In some embodiments, the object may include a biological object and/or a non-biological object. The biological object may include a human being, an animal, a plant, or a specific portion, organ, and/or tissue thereof. In some embodiments, the object may include a specific part of the body, such as a head, a chest, an abdomen, or the like, or any combination thereof. In some embodiments, the object may include a specific organ, such as a heart, a breast, an esophagus, a trachea, bronchus, a stomach, a gallbladder, a small intestine, a colon, a bladder, a ureter, a uterine, a tubal, etc. In some embodiments, when the object needs to be scanned, the object may be placed on an examination table 116 and may be moved with the examination table 116 along a transverse direction and/or a longitudinal direction of the medical device 110 to enter a scanning region 115. After the object enters the scanning region 115, the medical device 110 may perform an operation on the object based on a scanning protocol, and a corresponding medical image of the object may be obtained.

In some embodiments, the medical device 110 may include a single-mode scanning device and/or a multi-mode scanning device. The single-mode scanning device may include, for example, a computed tomography (CT) scanning device, a positron emission computed tomography (PET) scanning device, a magnetic resonance imaging (MRI) scanning device, a radio therapy (RT) device, an X-ray scanning device, etc. The multi-mode scanning device may include, for example, an X-ray imaging-magnetic resonance imaging (X-ray MRI) scanning device, a positron emission tomography X-ray imaging (PET-X-ray) scanning device, a single photon emission computed tomography magnetic resonance imaging (SPECT-MRI) scanning device, a positron emission tomography computer tomography (PET-CT) scanning device, etc. The scanning device provided above is merely for illustration and is not intended to limit the scope of the present disclosure. As used herein, the term “imaging mode” or “mode” may broadly refer to an imaging method or technology that collects, generates, processes, and/or analyzes the imaging information of a target object.

The processing device 120 may process data and/or information obtained from the medical device 110, the terminal device 130, the display device 140, and/or the storage device 150. For example, the processing device 120 may obtain a call instruction of virtual caregiver information, and direct the display device to display the virtual caregiver information based on the call instruction.

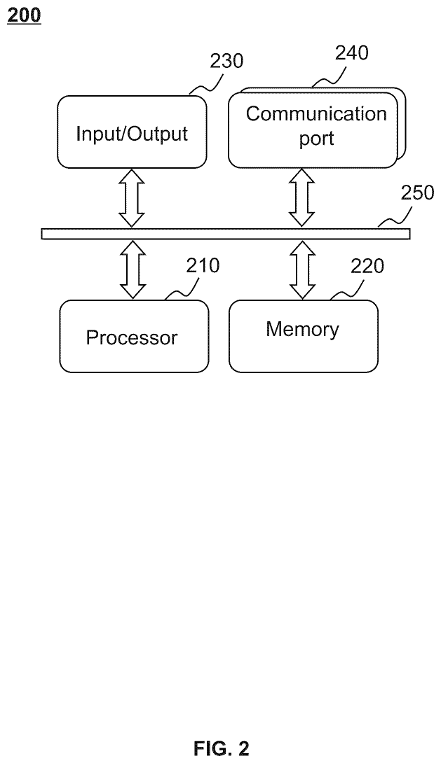

In some embodiments, the processing device 120 may be a single server or a group of servers. The group of servers may be centralized or distributed. In some embodiments, the processing device 120 may be local or remote. For example, the processing device 120 may access information and/or data stored in or acquired by the medical device 110, the terminal device 130, the display device 140, and/or the storage device 150 via the network 160. For another example, the processing device 120 may be directly connected to the medical device 110, the terminal device 130, the display device 140, and/or the storage device 150 to access stored or acquired information and/or data. In some embodiments, the processing device 120 may be implemented on a cloud platform. Merely by way of example, the cloud platform may include private cloud, public cloud, hybrid cloud, community cloud, distributed cloud, internal cloud, multi-cloud, or the like, or any combination thereof. In some embodiments, the processing device 120 may be implemented on a computing device 200 including one or more components shown in FIG. 2 in this specification.

The terminal device 130 may input/output signals, data, information, etc. In some embodiments, the terminal device 130 may include a mobile device 130-1, a tablet computer 130-2, a notebook computer 130-3, or any combination thereof. In some embodiments, the mobile device 130-1 may include a home device, a wearable device, a virtual reality (VR) device, an augmented reality device, or the like, or any combination thereof. The home device may include a lighting device, a control device of an intelligent electrical apparatus, a monitoring device, a television, a video camera, an interphone, or the like, or any combination thereof. The wearable device may include a bracelet, a footgear, eyeglasses, a helmet, a watch, clothing, a backpack, an accessory, or the like, or any combination thereof. The virtual reality device and/or the augmented reality device may include a virtual reality helmet, virtual reality glasses, a virtual reality patch, an augmented reality helmet, augmented reality glasses, an augmented reality patch, or the like, or any combination thereof. For example, the virtual reality device and/or the augmented reality device may include a Google Glass™, an Oculus Rift™, a Hololens™, a Gear VR™, etc.

In some embodiments, the terminal device 130 may include an operation terminal device (also referred to a first terminal device) and a control terminal device (also referred to a second terminal device). In some embodiments, a user of the operation terminal device may be an object, and a user of the control terminal device may be a medical person. In some embodiments, the operation terminal device and the control terminal device may be located in different physical spaces. For example, the control terminal device may be in a first space, and the operation terminal device may be in a second space. In some embodiments, the control terminal device and the operation terminal device may communicate with the processing device 120 through the network 160. For example, the processing device 120 may obtain twin data associated with the object from the operation terminal device, and transmit the twin data associated with the object to the control terminal device. The control terminal device may transmit guidance data to the processing device 120 based on the twin data associated with the object. The processing device 120 may obtain the guidance data from the control terminal device, and direct a display device associated with the operation terminal device to present the guidance data. As another example, the processing device 120 may obtain a control instruction from the control terminal device, and transmit the control instruction to the operation terminal device to control a medical device corresponding to the operation terminal device to control the medical operation.

In some embodiments, the terminal device 130 may interact with other components in the medical system 100 via the network 160. In some embodiments, a user (e.g., a doctor, the object) may interact with the medical device 110 and/or the processing device 120 through the terminal device 130. For example, the processing device 120 may obtain twin data associated with the object from the terminal device 130 (e.g., the operation terminal device). As another example, the terminal device 130 (e.g., the control terminal device) may obtain a user's (e.g., a user of the terminal device 130, such as a doctor) input information (e.g., guidance data, a control instruction) through an input device (e.g., a keyboard, a touch screen, a brain wave monitoring device), and transmit the input information to the processing device 120 for further processing.

In some embodiments, the terminal device 130 may be a portion of the processing device 120. For example, the terminal device 130 and the processing device 120 may be integrated as a control device of the medical device 110. In some embodiments, the terminal device 130 may be omitted.

The display device 140 may display data obtained from the medical device 110, the processing device 120, and/or the terminal device 130. For example, the display device 140 may display the virtual caregiver information. For another example, the display device 140 may display the guidance data. In some embodiments, the display device 140 may include a liquid crystal display (LCD), a light-emitting diode (LED)-based display, a flat panel display, a curved screen, a television device, a cathode ray tube (CRT), or the like, or a combination thereof. In some embodiments, the display device 140 may be a portion of the terminal device 130. For example, the display device 140 may be a viewing screen of the terminal device 130 (e.g., the wearable device, the VR device).

The storage device 150 may store data and/or instructions. In some embodiments, the storage device 150 may store data obtained from the medical device 110, the processing device 120, the terminal device 130, and/or the display device 140. For example, the storage device 150 may store reference information associated with a medical operation performed on the object. In some embodiments, the storage device 150 may store data and/or instructions that the processing device 120 may execute or use to execute the exemplary methods described in the present disclosure. For example, the storage device 150 may store instructions of the processing device 120 to execute the methods shown in the flowcharts in the present disclosure. In some embodiments, the storage device 150 may include a mass storage device, a removable storage device, a volatile read-write memory, a read-only memory (ROM), or the like, or any combination thereof. In some embodiments, the storage device 150 may be implemented on a cloud platform. In some embodiments, the storage device 150 may be a portion of the processing device 120.

The network 160 may include any suitable network that facilitates the exchange of information and/or data of the medical system 100. In some embodiments, one or more components of the medical system 100 may transmit information and/or data with one or more other components of the medical system 100 via the network 160. In some embodiments, the network 160 may be any one or more of a wired or wireless network. In some embodiments, the network may be point-to-point, shared, central and other topologies or a combination of a plurality of topologies. In some embodiments, the network 160 may include one or more network access points. For example, network 160 may include wired or wireless network access points.

In some embodiments, the medical system 100 may also include an acquisition device (not shown). The acquisition device may be used to acquire a video, an audio, an image, etc., including a user (e.g., the doctor, the object) and/or a device (e.g., the medical device 110). In some embodiments, the acquisition device may include a camera (e.g., a digital camera, an analog camera), a red-green-blue (RGB) sensor, an RGB depth (RGB-D) sensor, a recorder, or the like, or a combination thereof. In some embodiments, the acquisition device may be installed in a scanning room. For example, the acquisition device may be fixedly or slidably installed at a position such as a ceiling, a floor, a wall, etc. of the scanning room. In some embodiments, the acquisition device may be installed on the medical device 110. For example, the acquisition device may be installed on a frame of the medical device 110 or on a component such as the examination table 116 by means of fixing, sliding, and/or rotating.

In some embodiments, the medical system 100 may also include a medical auxiliary device (not shown) used for acquiring a physiological signal (e.g., an electrocardiographic signal, a respiratory signal, a brain wave signal) of the object. In some embodiments, the medical auxiliary device may include an electrocardiographic signal acquisition device, a respiratory signal acquisition device, a brain wave signal acquisition device, or the like, or any combination thereof. In some embodiments, the medical auxiliary device may be a part of the medical device 110. In some embodiments, the medical auxiliary device may be independent of the medical device 110.

It should be noted that the above description of the medical system 100 is merely for illustration, and does not limit the scope of application of the present disclosure. For those skilled in the art, various modifications and changes can be made according to the description of the present disclosure. For example, components of the medical system 100 may have their own storage device, or may use a common storage device. However, these modifications and changes may not deviate from the scope of the present disclosure.

FIG. 2 is a schematic diagram illustrating exemplary hardware and/or software components of an exemplary computing device according to some embodiments of the present disclosure. As shown in FIG. 2, in some embodiments, the computing device 200 may include a processor 210, a memory 220, an input/output (I/O) 230, and a communication port 240 connected via a bus 250.

The processor 210 may execute computer instructions (e.g., program code) and perform functions of the processing device 120 according to the method(s) described herein. The computer instructions may include, for example, routines, programs, objects, components, data structures, procedures, modules, and functions, which perform particular functions described herein. For example, the processor 210 may process data of the medical device 110, the terminal device 130, the storage device 150, and/or any other component in the medical system 100. In some embodiments, the processor 210 may include at least one hardware processor, such as a microcontroller, microprocessor, reduced instruction set computer (RISC), application specific integrated circuit (ASIC), application specific instruction set processor (ASIP), central processing unit (CPU), graphics processing unit (GPU), physical processing unit (PPU), microcontroller unit, digital signal processor (DSP), field programmable gate array (FPGA), high-order RISC Machine (ARM), programmable logic device (PLD), any circuit or processor or similar capable of performing at least one function, or any combination thereof.

Merely for illustration, only one processor is described in the computing device 200. However, it should be noted that the computing device 200 in the present disclosure may also include multiple processors, thus operations and/or method steps that are performed by one processor as described in the present disclosure may also be jointly or separately performed by the multiple processors. For example, if in the present disclosure the processor of the computing device 200 executes both operations A and B, it should be understood that operation A and operation B may also be performed by two or more different processors jointly or separately in the computing device 200 (e.g., a first processor executes operation A and a second processor executes operation B, or the first and second processors jointly execute operations A and B).

The memory 220 may store data/information obtained from the medical device 110, the terminal device 130, the storage device 150, and/or any other component in the medical system 100. In some embodiments, the memory 220 may include a mass storage, a removable storage, a volatile read-write memory, a read-only memory (ROM), or any combination thereof. In some embodiments, the memory 220 may store at least one program and/or instruction for executing the exemplary manner described in the present disclosure.

The input/output (I/O) 230 may be used to input and/or output signal, data, information, etc. In some embodiments, the input/output (I/O) 230 may enable the user to interact with processing device 120. In some embodiments, the input/output (I/O) 230 may include an input device and an output device. The input device may include a keyboard, a mouse, a touch screen, a microphone, or any combination thereof. The output device may include a display device, a speaker, a printer, a projector, or any combination thereof.

The communication port 240 may be connected to a network (e.g., the network 160) to facilitate data communication. The communication port 240 may establish a connection between the processing device 120 and the medical device 110, the terminal device 130, the display device 140, and/or the storage device 150. The connection may include a wired connection and a wireless connection. In some embodiments, the communication port 240 may be and/or include a standardized communication port, such as RS232, RS485, etc. In some embodiments, the communication port 240 may be a specially designed communication port. For example, the communication port 240 may be designed according to a digital imaging and medical communication (DICOM) protocol.

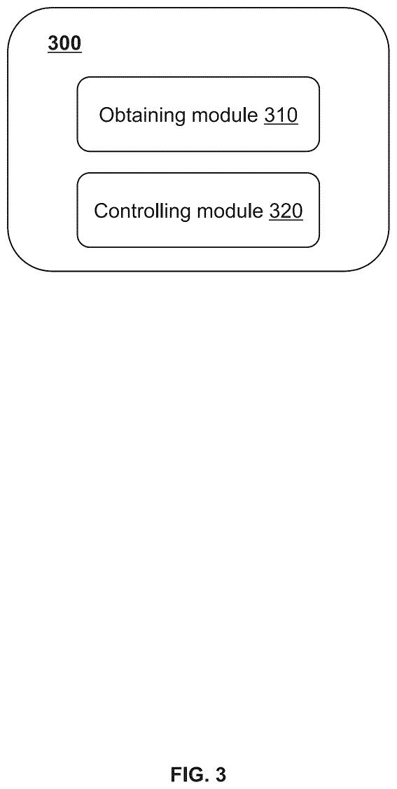

FIG. 3 is a block diagram illustrating an exemplary processing device according to some embodiments of the present disclosure. As shown in FIG. 3, in some embodiments, the processing device 120 may include an obtaining module 310 and a controlling module 320.

The obtaining module 310 may be configured to obtain an instruction with respect to reference information associated with a medical operation performed on an object. In some embodiments, the reference information may include an operational parameter of the medical operation.

In some embodiments, the obtaining module 310 may obtain a call instruction of the virtual caregiver information. In some embodiments, the obtaining module 310 may obtain a control instruction associated with the operational parameter.

In some embodiments, the obtaining module 310 may obtain feature information of the object, and determine state information of the object based on the feature information using a state determination model. In some embodiments, the obtaining module 310 may determine the instruction with respect to the reference information associated with the medical operation based on the state information.

The controlling module 320 may be configured to control the medical operation based on the instruction with respect to the reference information.

In some embodiments, the controlling module 320 may direct, based on the call instruction, a display device to display the virtual caregiver information. In some embodiments, the controlling module 320 may adjust, based on the control instruction, the operational parameter of the object.

It should be understood that the systems and modules shown in FIG. 3 may be implemented in various ways. For example, the obtaining module 310 and/or the controlling module 320 may be implemented entirely by hardware, software, or by combining software and hardware implementation. As another example, the obtaining module 310 and/or the controlling module 320 may share a processor and a non-transitory storage medium or have their own processors and non-transitory storage mediums. The non-transitory storage medium may store a computer program. When the processor executes the computer program, a corresponding function may be implemented.

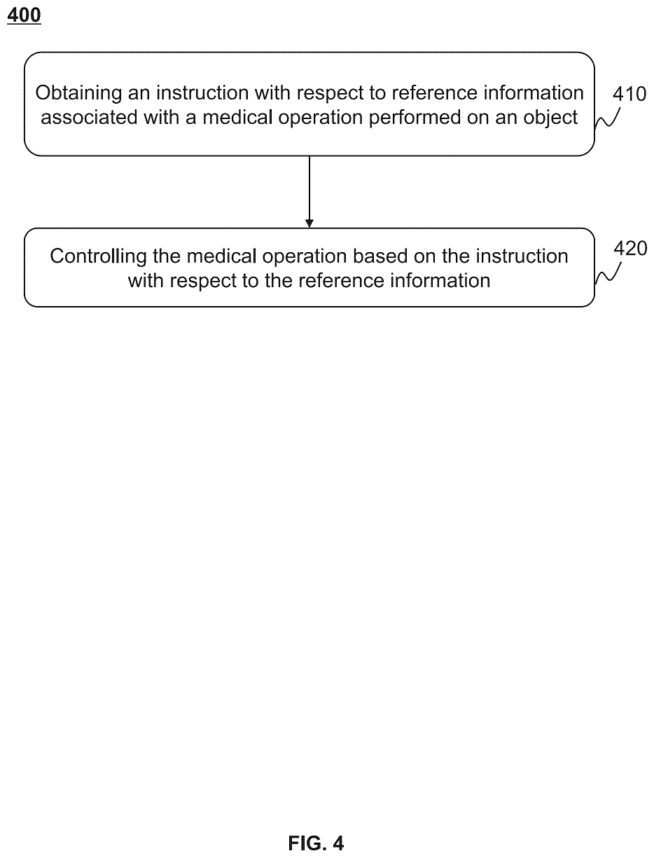

FIG. 4 is a flowchart illustrating an exemplary process for controlling a medical operation according to some embodiments of the present disclosure. In some embodiments, the process 400 may be executed by the medical system 100. For example, the process 400 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 400. The operations of the illustrated process presented below are intended to be illustrative. In some embodiments, the process 400 may be accomplished with one or more additional operations not described and/or Without one or more of the operations discussed. Additionally, the order of the operations of process 400 illustrated in FIG. 4 and described below is not intended to be limiting.

In 410, the processing device 120 (e.g., the obtaining module 310, the processor 210) may obtain an instruction with respect to reference information associated with a medical operation performed on an object.

In some embodiments, the medical operation may include, but is not limited to, a CT scanning, a magnetic resonance (MR) scanning, a PET scanning, a PET-CT scanning, radiotherapy, or the like, or any combination thereof. In some embodiments, the medical operation may include an operation associated with the CT scanning, the MR scanning, the PET scanning, the PET-CT scanning, radiotherapy, or the like, or any combination thereof, for example, setting or adjusting an operational parameter, controlling an operational procedure, controlling a related device, or the like, or any combination thereof. In some embodiments, the medical operation may be any medical operation associated with a medical procedure such as imaging, treatment, which is not limited in the present disclosure.

In some embodiments, the reference information may include the operational parameter of the medical operation.

The operational parameter refers to a parameter associated with the medical operation. In some embodiments, the operational parameter may include a scanning time, a scanning sequence (e.g., a sequence type, a sequence parameter), a scanning site, a scanning speed, a scanning intensity, a system parameter of the medical device, or the like, or any combination thereof. In some embodiments, the operational parameter of the medical operation may be preset. In some embodiments, the processing device 120 may determine the operational parameter based on basic information (e.g., an age, a gender, a height, a weight) and/or disease information (e.g., target volume information, organ-at-risk information) of the object. In some embodiments, the processing device 120 may determine the operational parameter based on a treatment plan or a scanning plan of the object. In some embodiments, the processing device 120 may determine the operational parameter based on state information (e.g., a physiological state, an emotional state) of the object.

In some embodiments, the instruction with respect to the reference information may include an instruction with respect to the virtual caregiver information, an instruction with respect to the operational parameter of the medical operation, or the like, or any combination thereof. In some embodiments, the instruction with respect to the reference information may include a call instruction of the virtual caregiver information, a control instruction associated with the operational parameter of the medical operation, or the like, or any combination thereof.

The virtual caregiver information may include pre-generated or real-time generated accompany information associated with a caregiver (e.g., a family member or a friend of the object). In some embodiments, the accompany information may include visual information, auditory information, tactile information, or the like, or any combination thereof. For example, the virtual caregiver information may be a pre-recorded video or audio of the caregiver. As another example, the virtual caregiver information may be caregiver information generated by real-time simulation.

In some embodiments, the reference information may further include a medical protocol (e.g., a scanning protocol, a radiotherapy protocol). Accordingly, the instruction with respect to the reference information may include a call instruction of the medical protocol. In some embodiments, the medical protocol may include a technical principle, a medical process introduction, a medical process duration, medical process precautions, or the like, or any combination thereof. For example, the scanning plan may include a scanning technical principle, a scanning process introduction, a scanning duration, scanning precautions, or the like, or any combination thereof. In some embodiments, a form of the medical protocol may include video information (e.g., VR information), audio information, image information, or the like, or any combination thereof. In some embodiments, the medical protocol may be generated in advance or in real time. For example, the processing device 120 may obtain a pre-generated medical protocol (e.g., the video information) from the storage device 150 according to a disease of the object. As another example, the processing device 120 may obtain the pre-generated medical protocol (e.g., the video information) from the storage device 150 based on the treatment plan or the scanning plan of the object. As still another example, the processing device 120 may determine a corresponding medical protocol in real time according to information of the object such as disease, age, gender, etc.

In some embodiments, the processing device 120 may obtain the instruction with respect to the reference information from the medical device 110 or the terminal device 130. For example, the object may initiate the call instruction of virtual caregiver information through the terminal device 130, and the processing device 120 may read the call instruction of virtual caregiver information from the terminal device 130 through a network (e.g., the network 160).

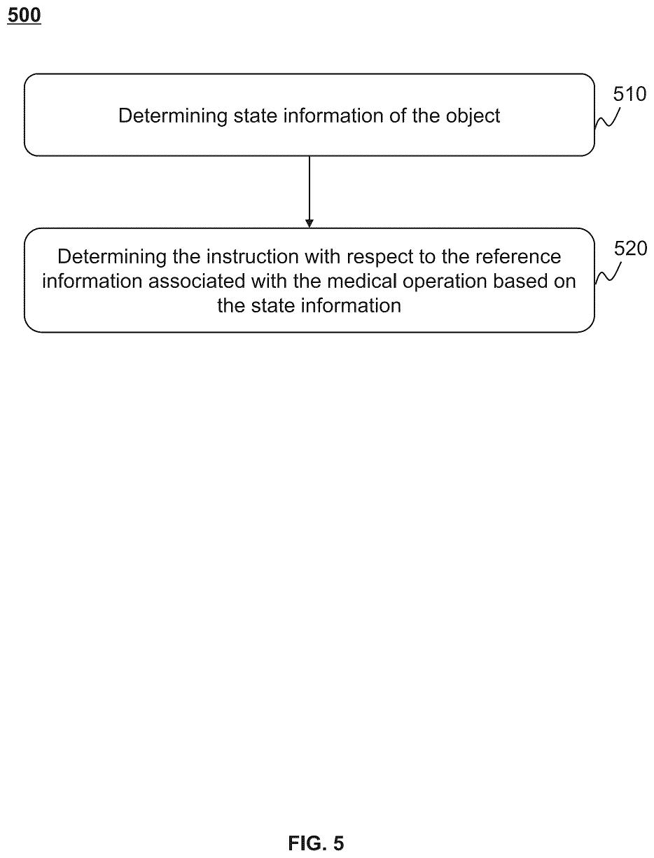

In some embodiments, the processing device 120 may determine the instruction with respect to the reference information associated with the medical operation based on state information of the object. In some embodiments, the state information of the object may include at least one of an emotional state, a physiological state, or a gaze state of the object. For example, the processing device 120 may determine the call instruction of the virtual caregiver information and/or the control instruction associated with the operational parameter of the medical operation based on the emotional state of the object. More descriptions may be found elsewhere in the present disclosure, for example, FIG. 5 and descriptions thereof.

In 420, the processing device 120 (e.g., the controlling module 320, the processor 210) may control the medical operation based on the instruction with respect to the reference information.

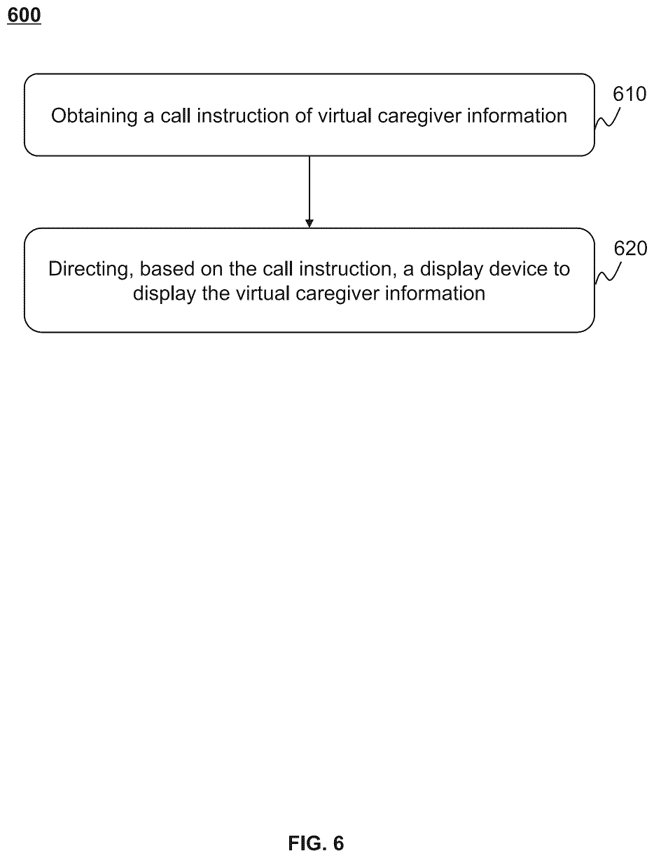

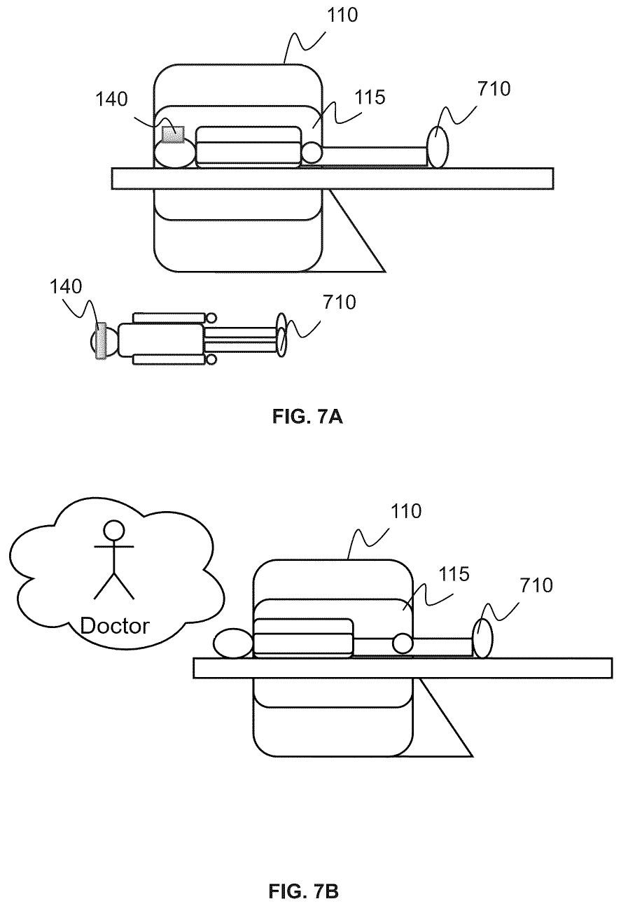

In some embodiments, as described in connection with above, the processing device 120 may direct a display device (e.g., the display device 140) to display the virtual caregiver information based on the call instruction of the virtual caregiver information. For example, before or during the medical operation, the processing device 120 may direct the display device 140 to display the virtual caregiver information. More descriptions may be found elsewhere in the present disclosure, for example, FIGS. 6 and 7 and the relevant descriptions thereof.

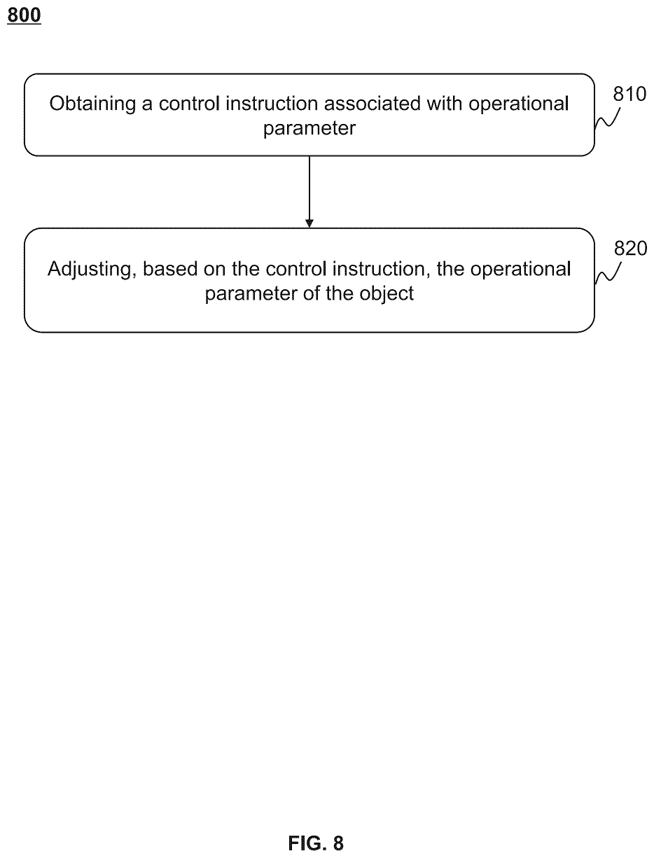

In some embodiments, as described in connection with above, the processing device 120 may adjust the operational parameter of the medical operation based on the control instruction associated with the operational parameter. For example, the processing device 120 may adjust the operational parameter of the medical operation before or during the medical operation. More descriptions may be found elsewhere in the present disclosure, for example, FIG. 8 and the descriptions thereof.

In some embodiments, as described in connection with above, the processing device 120 may obtain a corresponding medical protocol based on the call instruction of the medical protocol. In some embodiments, the processing device 120 may generate the corresponding medical protocol in real time based on the call instruction of the medical protocol. In some embodiments, the processing device 120 may obtain the pre-generated medical protocol from a storage device (e.g., the storage device 150) based on the call instruction of the medical protocol.

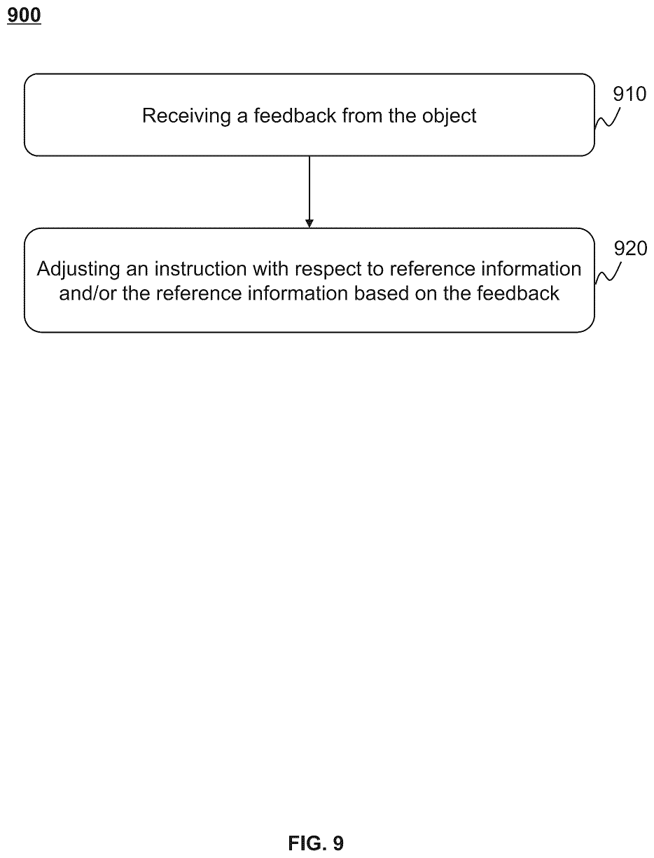

In some embodiments, the processing device 120 may receive a feedback from the object (e.g., via the terminal device 130) and adjust the instruction and/or the reference information based on the feedback from the object. For example, before or during the medical operation, the processing device 120 may receive the feedback from the object and adjust the reference information and/or the instruction with respect to the reference information accordingly. More descriptions may be found elsewhere in the present disclosure, for example, FIG. 9 and the descriptions thereof.

In some embodiments, before the medical operation, the processing device 120 may obtain twin data associated with the object from an operation terminal device (e.g., a terminal device used by the object), transmit the twin data associated with the object to a control terminal device (e.g., a terminal device used by a medical person), obtain guidance data from the control terminal device, and direct a display device (e.g., the display device 140) associated with the operation terminal device to present the guidance data, so as to guide the object to perform a preparatory operation associated with the medical operation (e.g., a medical scanning or a radiotherapy operation). More descriptions may be found elsewhere in the present disclosure, for example, FIG. 11 and the descriptions thereof.

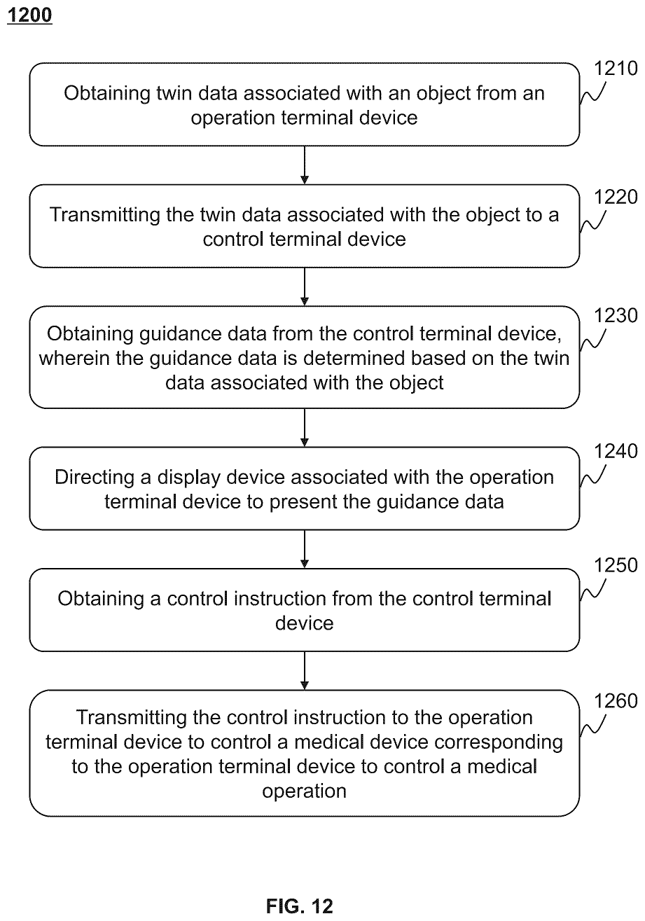

In some embodiments, the processing device 120 may obtain a control instruction from the control terminal device and transmit the control instruction to the operation terminal device to control a medical device corresponding to the operation terminal device to control the medical operation, so as to perform the medical operation on the object. In some embodiments, during the medical operation (e.g., during the medical scanning or the radiotherapy operation), the processing device 120 may obtain the control instruction from the control terminal device and transmit the control instruction to the operation terminal device to control the medical device corresponding to the operation terminal device to control the medical operation. More descriptions may be found elsewhere in the present disclosure, for example, FIG. 12 and the descriptions thereof.

It should be noted that the above description regarding the process 400 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 400 may be accomplished with one or more additional operations not described and/or without one or more of the operations discussed above.

FIG. 5 is a flowchart illustrating an exemplary process for determining an instruction with respect to reference information associated with a medical operation according to some embodiments of the present disclosure. In some embodiments, process 500 may be executed by the medical system 100. For example, the process 500 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting.

In 510, the processing device 120 (e.g., the obtaining module 310, the processor 210) may determine state information of the object.

The state information may reflect a current state of the object. In some embodiments, the state information may include an emotional state, a physiological state, a gaze state of the object, or the like, or a combination thereof.

The emotional state may reflect an emotion of the object, for example, anxiety, tension, fear, sadness, calm, etc. For example, for an object with claustrophobia, during a medical scanning of the head, since the head is located in a semi-enclosed scanning area, the object may experience the emotion such as fear or anxiety. As another example, a child or an object undergoing the medical scanning for the first time may have the emotion such as tension or fear before the medical scanning. As still another example, for an object who has experienced medical scanning many times, the object may be in the calm emotional state from the beginning to the end of the medical operation.

The physiological state may reflect physiological information of the object, for example, an electrocardiographic (ECG) signal, a respiratory signal, a brain wave signal, a heart rate, a blood oxygen, a blood pressure, etc. For example, for an object with a heart disease, fluctuation of the ECG signal at a certain moment or time period may be greater than a standard value. As another example, when the object is in a panic state, a waveform of the respiratory signal or the brain wave signal may have a large deviation from a preset waveform. As still another example, the heart rate and/or the blood pressure of an overstressed object may be greater than a preset value. In some embodiments, the physiological state may be expressed as a waveform (e.g., a signal waveform), a value (e.g., a blood pressure value), a value range, or the like, or a combination thereof.

The gaze state may reflect an eye gaze direction of the object, for example, left, right, up, down, an angle between the gaze direction and a horizontal or vertical direction, etc. For example, before or during the medical operation, the object may gaze a specific point or a specific direction which may indicate the object's interest for different virtual caregiver information or different operational parameters.

In some embodiments, the processing device 120 may determine the state information of the object using a state determination model. In some embodiments, the processing device 120 may obtain feature information of the object, and determine the state information of the object based on the feature information using the state determination model.

In some embodiments, the feature information may include an image, a video, an audio, a physiological signal associated with the object, or the like, or a combination thereof. In some embodiments, as described in connection with FIG. 1, the processing device 120 may obtain the feature information from the acquisition device (e.g., a camera).

In some embodiments, the processing device 120 may input the feature information into the state determination model to obtain the state information of the object. For example, the state determination model may output the emotional state of the object by analyzing a facial expression of the object in the image. As another example, the state determination model may output the gaze state of the object by analyzing a direction of the eyeball of the object in the image. As still another example, the state determination model may output the physiological state of the object by analyzing the physiological signal of the object.

In some embodiments, the state determination model may include a convolutional neural network (CNN) model, a fully connected neural network model, a recurrent neural network (RNN) model, etc. In some embodiments, the state determination model may include an emotional state determination model, a gaze state determination model, and a physiological state determination model. In some embodiments, the emotional state determination model, the gaze state determination model, and the physiological state determination model may be three submodules of the state determination model, or three independent models.

In some embodiments, the state determination model may be obtained through training based on a plurality of training samples. For example, the processing device 120 may obtain a plurality of first samples (e.g., sample images) labeled with the emotional state, train an initial machine learning model using the plurality of first samples, and obtain a trained emotional state determination model. As another example, the processing device 120 may obtain a plurality of second samples (e.g., sample images) labeled with the gaze state, train an initial machine learning model using the plurality of second samples, and obtain the trained gaze state determination model. As still another example, the processing device 120 may obtain a plurality of third samples (e.g., a plurality of sets of physiological signals) labeled with the physiological state, train an initial machine learning model using the plurality of third samples, and obtain the trained physiological state determination model. In some embodiments, the emotional state determination model, the gaze state determination model, and the physiological state determination model may be trained individually or jointly. For example, the initial machine learning model may be jointly trained using the first training samples, the second training samples, and the third training samples, so as to obtain the trained state determination model.

In some embodiments, the processing device 120 may determine the state information of the object in other ways, which is not limited in the present disclosure.

In some embodiments, the processing device 120 may obtain the state information of the object continuously or according to an interval. For example, the medical auxiliary device may monitor the brain wave of the object in real time, and the processing device 120 may read the brain wave signal of the object from the medical auxiliary device in real time or according to a specific time interval. As another example, the acquisition device may obtain an image including the eyes of the object in real time, and the processing device 120 may read the image from the acquisition device in real time or according to a specific time interval, so as to determine the eye gaze direction of the object. As still another example, the processing device 120 may read the image containing the object from the acquisition device before performing scanning or radiotherapy on the object, and obtain the emotional state of the object by analyzing and processing the image (e.g., inputting the image into the trained emotion state determination model).

In 520, the processing device 120 (e.g., the processor 210) may determine the instruction with respect to the reference information associated with the medical operation based on the state information.

In some embodiments, the processing device 120 may determine a call instruction of the virtual caregiver information based on the state information of the object.

In some embodiments, the processing device 120 may determine the call instruction of the virtual caregiver information based on the physiological state of the object. In some embodiments, the processing device 120 may determine virtual caregiver information corresponding to (or matched with) the physiological state and determine the call instruction of the virtual caregiver information. In some embodiments, different physiological states may correspond to different virtual caregiver information. In some embodiments, the physiological state and the virtual caregiver information may have a one-to-one correspondence or a matching relationship. In some embodiments, different physiological states (e.g., different waveforms, different values) may correspond to virtual caregiver information of different people (e.g., a parent, a wife, a husband, a child, a friend, a medical worker), virtual caregiver information of different types (e.g., an audio, a video, an image), virtual caregiver information of different forms (e.g., behavior, tone, tone of voice), etc.

For example, taking the ECG signal as an example, a first waveform, a second waveform, and a third waveform corresponding to different amplitudes, different periods, and/or different shapes may be determined based on historical ECG signals of a plurality of different objects. Further, the first waveform corresponds to virtual caregiver information of an immediate family member (e.g., a parent, a wife, a husband, a sibling, a child), the second waveform corresponds to virtual caregiver information of a collateral relative (e.g., a male cousin, a female cousin, an uncle) and/or a friend, and the third waveform corresponds to virtual caregiver information of a medical worker; or the first waveform corresponds to virtual caregiver information of the video category, the second waveform corresponds to virtual caregiver information of the audio category, and the third waveform corresponds to virtual caregiver information of the image category.

As another example, taking the respiratory signal as an example, if the signal amplitude is smaller than or equal to a first preset threshold, the signal may correspond to the virtual caregiver information of the friend or the medical worker and/or the virtual caregiver information of the image category; if the signal amplitude is greater than the first preset threshold and smaller than a second preset threshold, the signal may correspond to the virtual caregiver information of the parent and/or the virtual caregiver information of the audio category; if the signal amplitude is greater than or equal to the second preset threshold, the signal may correspond to the virtual caregiver information of the wife, the husband, or the child, and/or the virtual caregiver information of the video category.

In some embodiments, the processing device 120 may compare the physiological signal (e.g., the respiratory signal, the ECG signal, the brain wave signal) reflecting the physiological state of the object with the preset waveforms corresponding to the physiological signal and determine the virtual caregiver information corresponding to (or matched with) the physiological state. For example, the processing device 120 may compare the real-time ECG signal of the object with the preset waveforms (e.g., the first waveform, the second waveform, and the third waveform) corresponding to the ECG signal, determine the preset waveform matched with (e.g., with the same, substantially the same, or similar signal amplitude, period, and/or shape) the ECG signal of the object, and determine the virtual caregiver information corresponding to the matched waveform as target virtual caregiver information of the object.

In some embodiments, the processing device 120 may compare the physiological signal (e.g., the respiratory signal, the ECG signal, the brain wave signal) reflecting the physiological state of the object with preset signal threshold(s) corresponding to the physiological signal and determine the virtual caregiver information corresponding to (or matched with) the physiological state. For example, the processing device 120 may compare the signal amplitude of the respiratory signal of the object with the preset signal thresholds corresponding to the respiratory signal; when the signal amplitude of the respiratory signal is smaller than or equal to the first preset threshold, the processing device 120 may determine the virtual caregiver information corresponding to the friend or the medical worker as the target virtual caregiver information; when the signal amplitude of the respiratory signal is greater than the first preset threshold and smaller than the second preset threshold, the processing device 120 may determine the virtual caregiver information corresponding to the parent as the target virtual caregiver information; when the signal amplitude of the respiratory signal is greater than or equal to the second preset threshold, the processing device 120 may determine the virtual caregiver information corresponding to the wife or the husband as the target virtual caregiver information.

In some embodiments, the processing device 120 may determine the target virtual caregiver information based on the physiological signal (or the physiological state) of the object in other ways, which is not limited in the present disclosure. For example, the processing device 120 may determine the target virtual caregiver information based on the physiological signal (or the physiological state) of the object using a trained accompany information determination model. Merely by way of example, the processing device 120 may input the physiological signal of the object, such as the ECG signal, the respiratory signal, the brain wave signal, the blood oxygen, the blood pressure, or the like, or any combination thereof, into the trained accompany information determination model, and the accompany information determination model may output the target virtual caregiver information of the object. In some embodiments, the accompany information determination model may be obtained through training based on sample data. For example, the processing device 120 may obtain a plurality of sets of historical physiological signals and historical virtual caregiver information corresponding to the plurality of sets of historical physiological signals, use the historical physiological signals as training inputs and the corresponding historical virtual caregiver information as training labels, train an initial machine learning model to obtain the trained accompany information determination model. In some embodiments, the accompany information determination model may include the CNN model, the fully connected neural network model, the RNN model, etc.

In some embodiments, the processing device 120 may determine the call instruction of the virtual caregiver information based on the gaze state of the object. In some embodiments, the processing device 120 may determine virtual caregiver information corresponding to (or matched with) the gaze state of the object and determine the call instruction of the virtual caregiver information. In some embodiments, different gaze states may correspond to different virtual caregiver information. In some embodiments, different gaze states (e.g., different gaze directions) may correspond to different points of interest (POIs) or regions of interest (ROIs) on a scanning cavity wall (or a display device), wherein the different points of interest (POIs) or regions of interest (ROIs) on the scanning cavity wall (or the display device) may correspond to virtual caregiver information of different people (e.g., a parent, a wife, a husband, a child, a friend, a medical worker), virtual caregiver information of different types (e.g., an audio, a video, an image), virtual caregiver information of different forms (e.g., behavior, tone, tone of voice), etc.

Merely by way of example, the processing device 120 may direct a projection device to project and display virtual caregiver information (or a summary or part of contents of the virtual caregiver information) of different people (e.g., a parent, a wife, a husband, a child, a friend, a medical worker), virtual caregiver information (or a summary or part of contents of the virtual caregiver information) of different types (e.g., an audio, a video, an image), virtual caregiver information (or a summary or part of contents of the virtual caregiver information) of different forms (e.g., behavior, tone, tone of voice), etc. on the scanning cavity wall (or the display device). Further, the processing device 120 may detect the eye gaze direction of the object in real time, determine the POI or ROI based on the gaze direction, and determine virtual caregiver information projected at the POI or ROI as the target virtual caregiver information, and further determine the call instruction of the target virtual caregiver information, so as to direct the display device (e.g., the display device 140) corresponding to the object to display the target virtual caregiver information.

In some embodiments, the processing device 120 may consider a duration of the gaze state of the object, only when the duration of the gaze state exceeds a time threshold, the processing device 120 may determine that POI or ROI and further determine the virtual caregiver information corresponding to the POI or ROI as the target virtual caregiver information.

In some embodiments, the processing device 120 may determine the call instruction of the virtual caregiver information based on the emotional state of the object. In some embodiments, the processing device 120 may determine virtual caregiver information corresponding to (or matched with) the emotional state and determine the call instruction of the virtual caregiver information. In some embodiments, as described in connection with above, the emotional state of the object may include anxiety, tension, fear, sadness, calm, etc. In some embodiments, the emotional state and the virtual caregiver information may have a one-to-one correspondence or a matching relationship. In some embodiments, different emotional states (e.g., different waveforms, different values) may correspond to virtual caregiver information of different people (e.g., a parent, a wife, a husband, a child, a friend, a medical worker), virtual caregiver information of different types (e.g., an audio, a video, an image), virtual caregiver information of different forms (e.g., behavior, tone, tone of voice), etc.

For example, the emotional state of fear may correspond to virtual caregiver information of the video category and/or virtual caregiver information of the immediate family member (e.g., the parent, the child); the emotional state of tension may correspond to virtual caregiver information of the audio category and/or virtual caregiver information of the friend; the emotional state of calm may correspond to virtual caregiver information of the image category, etc. Accordingly, when the object is in the emotional state of fear, the processing device 120 may read a video recorded in advance by the parent, determine the video as the target virtual caregiver information, and determine the call instruction of the target virtual caregiver information; when the object is in the emotional state of tension, the processing device 120 may read an audio recorded in advance or in real time by the friend, determine the audio as the target virtual caregiver information, and determine the call instruction of the target virtual caregiver information; when the object is in the emotional state of calm, the processing device 120 may read a plurality of images in advance taken by the friend, the relative, or the medical worker, determine the plurality of images as the target virtual caregiver information, and determine the call instruction of the target virtual caregiver information.

In some embodiments, when determining the target virtual caregiver information and/or the call instruction of the target virtual caregiver information, the processing device 130 may comprehensively the emotional state, the physiological state, and/or the gaze state.

According to the embodiments of the present disclosure, the state information such as the emotional state, the physiological state, and/or the gaze state (e.g., eye gaze direction) of the object may be monitored, the virtual caregiver information that matches the state information may be determined, and the call instruction corresponding to the virtual caregiver information may be determined, which can automatically select appropriate virtual caregiver information for the object and improve the medical experience of the object.

In some embodiments, the processing device 120 may determine the control instruction associated with the operational parameter based on the state information of the object.

In some embodiments, the processing device 120 may determine the control instruction associated with the operational parameter based on the emotional state of the object. In some embodiments, the processing device 120 may determine a target operational parameter and/or an adjustment manner of the target operational parameter based on the emotional state of the object and generate a corresponding control instruction. In some embodiments, different emotional states may correspond to different operational parameters (e.g., a scanning time, a scanning sequence, a scanning site, a scanning speed, a scanning intensity, a system parameter of the medical device) and/or different adjustment manners (e.g., increasing, decreasing, remaining unchanged).

For example, if the emotional state is calm, the operational parameter may remain unchanged. As another example, if the emotional state is tense or sad, the scanning rate may be increased by 20%, the scanning time may be decreased by 50%, the scanning intensity may be decreased by 10%, etc. As still another example, if the emotional state is anxiety, the scanning may be suspended, the scanning speed may be increased by 50%, the scanning time may be decreased by 70%, the scanning intensity may be decreased by 20%, etc. As still another example, if the emotional state is tense, the target operational parameter may be determined as an ordinary scanning sequence, free breathing, and under-sampling. As still another example, if the emotional state is calm, the target operational parameter may be determined as the ordinary scanning sequence, breath holding, and full-sampling. As still another example, if the emotional state is anxiety, the target operational parameter may be determined as a silent scanning sequence, free breathing, and under-sampling.

In some embodiments, the processing device 120 may determine the target operational parameter and/or the adjustment manner of the target operational parameter based on the emotional state of the object using a trained parameter determination model. For example, the processing device 120 may input the emotional state of the object into the trained parameter determination model, and the parameter determination model may output the target operational parameter and/or the adjustment manner of the target operational parameter corresponding to the emotional state. In some embodiments, the parameter determination model may be trained based on sample data. For example, the processing device 120 may obtain a plurality of sets of historical operational parameters and emotional states corresponding to the historical operational parameters, use the emotional states as training inputs and the historical operational parameters corresponding to the emotional states as labels, and train an initial machine learning model to obtain the trained parameter determination model. In some embodiments, the parameter determination model may include the CNN model, the fully connected neural network model, the RNN model, etc.

In some embodiments, the processing device 120 may determine the control instruction associated with the operational parameter based on the physiological state of the object. In some embodiments, the processing device 120 may determine the target operational parameter and/or the adjustment manner of the target operational parameter based on the physiological state of the object and generate a corresponding control instruction. In some embodiments, different physiological states may correspond to different operational parameters and/or different adjustment manners. In some embodiments, the physiological state and the target operational parameters and/or the adjustment manners of the target operational parameters may have a one-to-one correspondence or a matching relationship.

Merely by way of example, a first physiological signal, a second physiological signal, and a third physiological signal may respectively correspond to different amplitudes, accordingly, the scanning rate corresponding to a first physiological signal may be a first value, the scanning rate corresponding to a second physiological signal may be a second value, the scanning rate corresponding to a third physiological signal may be a third value, the greater the signal amplitude is, the greater the corresponding scanning rate may be.

In some embodiments, when determining the target operational parameter and/or the adjustment manner of the target operational parameter and/or the corresponding control instruction, the processing device 130 may comprehensively the emotional state and the physiological state.

In some embodiments, when determining the target operational parameter and/or the adjustment manner of the target operational parameter and/or the corresponding control instruction based on the state information of the object, the processing device 120 may also consider preset parameter range(s) (e.g., safe ranges). In some embodiments, different tissues/organs may correspond to different preset parameter ranges. For example, in a CT scanning, a window width of a soft tissue may range from 300 HU-400 HU and a window level of the soft tissue may range from 30 HU-60 HU; a window width of a bone may range from 1000 HU-1500 HU and a window level of the bone may range from 250 HU-350 HU. In some embodiments, under different emotional states and/or different physiological states, the preset parameter ranges may be correspondingly different.

According to the embodiments of the present disclosure, the control instruction associated with the operational parameter may be determined based on the state information of the object, which can avoid an inaccurate scanning result caused by the emotional or physiological abnormality of the object and reduce the damage of the scanning to the object.

It should be noted that the above description regarding the process 500 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 6 is a flowchart illustrating an exemplary process for providing virtual caregiver information during a medical operation according to some embodiments of the present disclosure. In some embodiments, process 600 may be executed by the medical system 100. For example, the process 600 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 600. The operations of the illustrated process presented below are intended to be illustrative. In some embodiments, the process 600 may be accomplished with one or more additional operations not described and/or without one or more of the operations discussed. Additionally, the order of the operations of process 600 illustrated in FIG. 6 and described below is not intended to be limiting.

In 610, the processing device 120 (e.g., the obtaining module 310, the processor 210) may obtain a call instruction of virtual caregiver information.

As described in process 400, the reference information may include the virtual caregiver information during the medical operation. Accordingly, the instruction may include the call instruction of the virtual caregiver information.

As described in connection with above, the virtual caregiver information may include visual information, auditory information, tactile information, or the like, or any combination thereof. The visual information may enable an object to see a caregiver through the display device 140 (e.g., a virtual reality (VR) wearable device). The auditory information may enable the object to hear sound (e.g., words of comfort and care) of the caregiver through the display device 140. The tactile information may enable the object to feel touch of the caregiver through the display device 140.

In some embodiments, the virtual caregiver information may be obtained in various ways. In some embodiments, the virtual caregiver information of the caregiver or a scanning technician may be obtained in real time or in advance using a sensor. In some embodiments, the sensor may include a visual sensor, an auditory sensor, a tactile sensor, or the like, or any combination thereof. For example, the VR video information of the caregiver may be recorded in real time through the visual sensor as the virtual caregiver information. As another example, at least one of the visual information, the auditory information, or the tactile information may be acquired by the sensor as the virtual caregiver information in a pre-acquisition manner. In some embodiments, the virtual caregiver information may be determined by simulation. For example, according to information (e.g., a height, a weight, an age, a gender, voice, image, video) of a target person (e.g., a relative, a friend, a medical worker of the object), at least one of the visual information, the auditory information, or the tactile information may be simulated and generated as the virtual caregiver information.

In some embodiments, as described in connection with FIG. 5, the processing device 120 may determine the call instruction of the virtual caregiver information based on state information of the object. For example, the processing device 120 may determine target virtual caregiver information corresponding to a physiological state and determine the call instruction of the target virtual caregiver information. As another example, the processing device 120 may determine target virtual caregiver information according to an eye gaze direction of the object and determine the call instruction of the target virtual caregiver information. As still another example, the processing device 120 may obtain target virtual caregiver information matching the emotional state of the object and determine the call instruction for the target virtual caregiver information.

In some embodiments, the processing device 120 may obtain the call instruction of the virtual caregiver information initiated by the object. In some embodiments, the processing device 120 may obtain the call instruction initiated by the object from a terminal device (e.g., the terminal device 130) or a medical device (e.g., the medical device 110). In some embodiments, the terminal device or the medical device may generate the call instruction based on an operation of the object on the device or an action of the object.

In some embodiments, the operation of the object on the device may include clicking, sliding, dragging, or the like, or any combination thereof. For example, the terminal device 130 may obtain a clicking operation of the object on a display screen, generate the call instruction of the virtual caregiver information based on the operation, and transmit the generated call instruction to the processing device 120.

In some embodiments, the action of the object may include a gesture (e.g., raising hands, waving hands), shaking the head, nodding the head, or the like, or any combination thereof. For example, the medical device 110 may obtain a gesture (e.g., a left-handed swinging, a right-handed swinging, a two-handed swinging) of the object in the scanning region 115, determine a corresponding operation instruction (e.g., the left-handed swinging means selecting content in a left area, the right-handed swinging means selecting content in a right area, the two-handed swinging means selecting content in a middle area) based on the gesture, generate the call instruction of the virtual caregiver information based on the operation instruction, and transmit the generated call instruction to the processing device 120.

In some embodiments, the object may initiate the call instruction before (e.g., when a head area of the object is moved into the scanning region 115 of the medical device 110) or during a medical operation.

In some embodiments, the processing device 120 may obtain the call instruction of the virtual caregiver information in other ways, for example, based on the voice of the object, which is not limited in the present disclosure.

In 620, the processing device 120 (e.g., the controlling module 320, the processor 210) may direct, based on the call instruction, a display device to display the virtual caregiver information.

In some embodiments, the processing device 120 may direct the display device worn by the object to display the virtual caregiver information. Merely by way of example, as shown in FIG. 7A, during a medical scanning or radiotherapy, when the head region of an object 710 is moved into the scanning region 115 of the medical device 110, the processing device 120 may direct the display device 140 (e.g., the display screen of the VR wearable device) to display the virtual caregiver information based on the call instruction (e.g., a call instruction determined based on the state information, a call instruction initiated by the object), so that the object can perceive the virtual caregiver information.

In some embodiments, the processing device 120 may direct the display device to display the virtual caregiver information on an inner wall of the medical device 110. For example, during the medical scanning or radiotherapy, when the head region of the object is moved into the scanning region 115 of the medical device 110, the processing device 120 may direct the display device 140 (e.g., an optical projection device) to project the virtual caregiver information onto the inner wall of the medical device 110 based on the call instruction, so that the object can receive the virtual caregiver information.

In some embodiments, the processing device 120 may direct the display device to display the virtual caregiver information outside a scanning cavity of the medical device 110. For example, as shown in FIG. 7B, during the medical scanning or radiotherapy, when the head region of the object 710 is moved outside the scanning region 115 of the medical device 110, the processing device 120 may direct the display device 140 (e.g., the optical projection device) to project the virtual caregiver information outside the scanning cavity of the medical device 110 based on the call instruction, so that the object can receive the virtual caregiver information.

According to the embodiments of the present disclosure, the virtual caregiver information may be called through the call instruction when the object needs to be accompanied and the display device (e.g., the VR wearable device worn by the object) may display the virtual caregiver information based on the call instruction, so as to realize the accompanying of the object and improve the accompanying effect of the object during scanning.

In some embodiments, before performing the medical operation (e.g., before displaying the virtual caregiver information or obtaining the call instruction of the virtual caregiver information), the processing device 120 may direct the display device to display a scanning protocol associated with the object based on the call instruction of the scanning protocol.

According to the embodiments of the present disclosure, before the medical operation is performed, the real-time recorded or pre-recorded information for introducing the medical operation may be displayed to the object through the display device, which can help the object better understand the medical operation, improve scanning efficiency, and improve medical experience of the object.

It should be noted that the above description regarding the process 600 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 600 may be accomplished with one or more additional operations not described and/or without one or more of the operations discussed above.

FIG. 8 is a flowchart illustrating an exemplary process for adjusting an operational parameter of a medical operation according to some embodiments of the present disclosure. In some embodiments, process 800 may be executed by the medical system 100. For example, the process 800 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 800. The operations of the illustrated process presented below are intended to be illustrative. In some embodiments, the process 800 may be accomplished with one or more additional operations not described and/or without one or more of the operations discussed. Additionally, the order of the operations of process 800 illustrated in FIG. 8 and described below is not intended to be limiting.

In 810, the processing device 120 (e.g., the obtaining module 310, the processor 210) may obtain a control instruction associated with the operational parameter.

As described in the process 400, the reference information may include the operational parameter of the medical operation. Accordingly, the instruction may include a control instruction associated with the operational parameter.

As described in connection with above, the operational parameter may include a scanning time, a scanning sequence, a scanning site, a scanning speed, a scanning intensity, a system parameter of the medical device, or the like, or any combination thereof.

In some embodiments, as described in connection with FIG. 5, the processing device 120 may determine the control instruction associated with the operational parameter based on state information of the object. For example, the processing device 120 may determine a target operational parameter based on an emotional state of the object and determine the control instruction associated with the target operational parameter. As another example, the processing device 120 may adjust an initial operational parameter based on a physiological state of the object and make the adjusted operational parameters fall within a preset parameter range.

In some embodiments, the processing device 120 may obtain the control instruction associated with the operational parameter initiated by the object or a doctor. In some embodiments, the processing device 120 may obtain the control instruction associated with the operational parameter initiated by the object or the doctor from a terminal device (e.g., the terminal device 130) or a medical device (e.g., the medical device 110). In some embodiments, the terminal device or the medical device may generate the control instruction based on an operation of the object or the doctor on the device (e.g., clicking, sliding, dragging), or an action of the object or the doctor (e.g., a gesture, shaking the head, nodding the head). For example, the terminal device 130 may obtain the gesture (e.g., a left-handed swinging, a right-handed swinging, a two-handed swinging) of the doctor or the object in the scanning region 115, determine a corresponding operation based on the gesture, generate a control instruction related to the operational parameter based on the operation instructions, and transmit the generated control instruction to the processing device 120. As another example, the doctor may input the control instruction associated with the operational parameter through a control terminal device, and the processing device 120 may read the corresponding control instruction from the control terminal device. As another example, the object may input the control instruction associated with the operational parameter through an operation terminal device, and the processing device 120 may read the corresponding control instruction from the operation terminal device. More descriptions regarding the control instruction initiated through the control terminal device and the operation terminal device may be found in FIG. 12 and the descriptions thereof.

In 820, the processing device 120 (e.g., the processor 210) may adjust, based on the control instruction, the operational parameter of the object.

In some embodiments, the processing device 120 may adjust the operational parameter of the object based on the control instruction, so as to control the medical device (e.g., the medical device 110) to perform a medical scanning or radiotherapy based on the adjusted operational parameter.

In some embodiments, the processing device 120 may adjust the operational parameter to a corresponding value and/or mode based on the control instruction. For example, the processing device 120 may set the operational parameter (e.g., a window width, a window level, a resolution, a scanning time) of the medical device to the corresponding value based on the control instruction. As another example, according to the control instruction, the processing device 120 may set the scanning sequence as a silent scanning sequence, a sampling sequence as a full sampling sequence, a scanning mode as a silent mode, etc.

In some embodiments, the processing device 120 may transmit the control instruction to the operation terminal device to control the medical device corresponding to the operation terminal device to control the medical operation.

It should be noted that the above description regarding the process 800 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 9 is a flowchart illustrating an exemplary process for adjusting an instruction with respect to reference information associated with a medical operation and/or the reference information according to some embodiments of the present disclosure. In some embodiments, process 900 may be executed by the medical system 100. For example, the process 900 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 900. The operations of the illustrated process presented below are intended to be illustrative. In some embodiments, the process 900 may be accomplished with one or more additional operations not described and/or without one or more of the operations discussed. Additionally, the order of the operations of process 900 illustrated in FIG. 9 and described below is not intended to be limiting.

In 910, the processing device 120 (e.g., the processor 210) may receive a feedback from the object.

The feedback may reflect feedback of the object on a medical operation, virtual caregiver information, or an operational parameter. In some embodiments, the feedback may include an instruction of the object for selecting or adjusting an operational parameter, virtual caregiver information, a medical protocol, etc. For example, the feedback may include an instruction of the object for selecting or adjusting a specific virtual caregiver information. As another example, the feedback may include an instruction of the object for selecting or adjusting the operational parameter.

In some embodiments, the processing device 120 may obtain the feedback from a terminal device (e.g., the terminal device 130). For example, the processing device 120 may obtain a feedback of the object on the virtual caregiver information, a scanning protocol, or the operational parameter from an operation terminal device. As another example, the processing device 120 may obtain a feedback of a doctor on the operational parameter from a control terminal device. In some embodiments, the feedback may include a voice, a gesture, an eye movement, a brain wave signal, a touch operation (e.g., clicking, sliding) through the terminal device 130, etc.

In some embodiments, the feedback may include a feedback of the virtual caregiver information and/or a feedback of the operational parameter.

In some embodiments, the feedback of the virtual caregiver information may include adjusting and/or selecting a display parameter (e.g., a playback speed, playback content, a volume, a background, a color, a resolution, a playback window size) of the virtual caregiver information. For example, when watching a VR video of the virtual caregiver information displayed on the display device 140, the object may select or adjust a video background of the VR video through a voice instruction. As another example, when listening an audio of the virtual caregiver information displayed on the display device 140, the object may increase a volume of the audio by moving the eyeball upwards and decrease the volume by moving the eyeball downward. As still another example, when watching an image of the virtual caregiver information displayed on the display device 140, the object may switch to a next image by turning left, right, down, or up.

In some embodiments, the feedback of the operational parameter may include adjusting and/or selecting the operational parameter (e.g., a scanning time, a scanning sequence (e.g., a sequence type, a sequence parameter), a scanning site, a scanning speed, a scanning intensity, a system parameter of the medical device). In some embodiments, the adjusting the operational parameter may include an action to adjust the operational parameter and/or a value that needs to be adjusted. In some embodiments, the adjusting the operating parameter may include a target value and/or a target mode of the operational parameter. Merely by way of example, the adjusting the operational parameter may include modifying an ordinary scanning sequence to a silent scanning sequence, modifying a full sampling sequence to an under-sampling sequence, modifying an ordinary sequence to a motion calibration sequence, etc. As another example, the adjusting the operational parameter may include adjusting a B value of a diffusion imaging sequence, adjusting a sampling rate, etc. As still another example, the adjusting the operational parameter may include adjusting a radio frequency pulse parameter so that a radio frequency parameter may correspond to a low specific absorption rate (SAR) value, a gradient parameter may correspond to a low PNS value, etc. As still yet another example, the adjusting the operational parameter may include adjusting the scanning speed, increasing ventilation of a system, reducing a temperature of a scanning room where a medical device is located, switching a field strength of a main magnetic field, adjusting the scanning mode from the ordinary mode to the silent mode, modifying breath holding to free breathing, etc.

In 920, the processing device 120 (e.g., the processor 210) may adjust the instruction with respect to reference information and/or the reference information based on the feedback.

In some embodiments, the processing device 120 may adjust the virtual caregiver information and/or the operational parameter based on the feedback. For example, after receiving the feedback sent by the object, the processing device 120 may direct a VR wearable device of the object to display the virtual caregiver information selected or adjusted by the object. As another example, after receiving the feedback sent by the object or the doctor, the processing device 120 may adjust the corresponding operational parameter to the target mode or the target value set by the object or the doctor.

In some embodiments, the processing device 120 may adjust a call instruction of the virtual caregiver information and/or a control instruction associated with the operational parameter based on the feedback. For example, after receiving the feedback from the object, the processing device 120 may generate a new call instruction of virtual caregiver information based on the virtual caregiver information selected or adjusted by the object. As another example, after receiving the feedback of the object or the doctor on the scanning mode, the processing device 120 may generate a control instruction corresponding to the scanning mode based on the feedback.

According to the embodiments of the present disclosure, when the object issues the feedback for selecting or adjusting the virtual caregiver information, corresponding virtual caregiver information may be provided in response to the feedback; when the object issues the feedback for adjusting or selecting the operational parameter, corresponding operational parameter may be adjusted or selected in response to the feedback, which can improve the medical experience of the object.

It should be noted that the above description regarding the process 900 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 900 may be accomplished with one or more additional operations not described and/or without one or more of the operations discussed above.

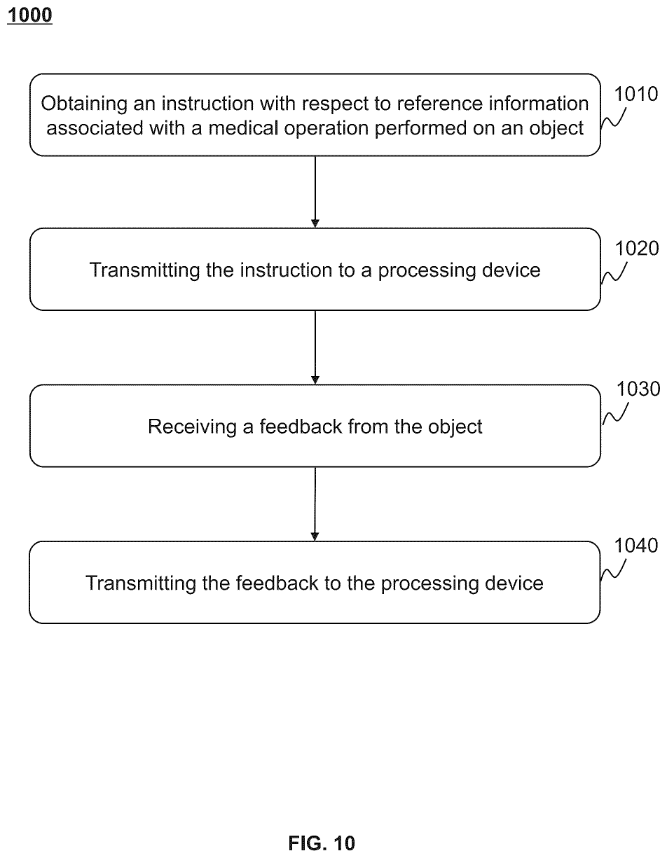

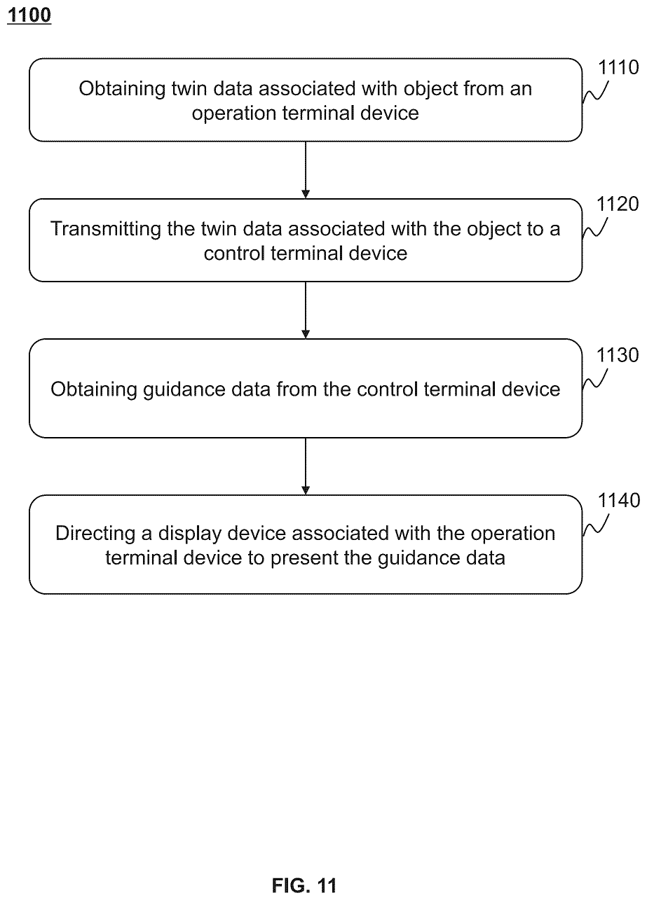

FIG. 10 is a flowchart illustrating an exemplary process for providing virtual caregiver information during a medical operation according to some embodiments of the present disclosure. In some embodiments, process 1000 may be executed by the medical system 100. For example, the process 1000 may be implemented as a set of instructions stored in the storage device (e.g., the storage device 150). In some embodiments, the terminal device 130 may execute the set of instructions and may accordingly be directed to perform the process 1000. The operations of the illustrated process presented below are intended to be illustrative. In some embodiments, the process 1000 may be accomplished with one or more additional operations not described and/or without one or more of the operations discussed. Additionally, the order of the operations of process 1000 illustrated in FIG. 10 and described below is not intended to be limiting.

In 1010, the terminal device 130 may obtain an instruction with respect to reference information associated with a medical operation performed on an object.

As described in connection with above, the instruction may include a call instruction of virtual caregiver information, a control instruction associated with an operational parameter of the medical operation, or the like, or any combination thereof. In some embodiments, the terminal device 130 may determine the instruction based on a user operation. For example, the user operation may include a gesture, a voice, an eye movement, a brain wave signal, a touch operation (e.g., clicking, sliding) through the terminal device 130, etc. More descriptions may be found in FIG. 4 and the descriptions thereof, which will not be repeated herein.

In 1020, the terminal device 130 may transmit the instruction to the processing device 120.

In some embodiments, after receiving the instruction, the processing device 120 may control the medical operation based on the received instruction. For example, the processing device 120 may direct a display device to display the virtual caregiver information based on the call instruction. More descriptions may be found in FIG. 6 and the descriptions thereof. As another example, the processing device 120 may adjust the operational parameter of the object based on the control instruction. More descriptions may be found in FIG. 8 and the descriptions thereof.

In 1030, the terminal device 130 may receive a feedback from the object.

More descriptions may be found in FIG. 9 and the descriptions thereof, which will not be repeated herein.

In 1040, the terminal device 130 may transmit the feedback to the processing device 120.

In some embodiments, after receiving the feedback, the processing device 120 may adjust the instruction with respect to the reference information and/or the reference information based on the feedback. More descriptions may be found in FIG. 9 and the descriptions thereof.

It should be noted that the above description regarding the processes 900 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Generally, the existing medical image acquisition manner requires a medical worker and a patient to be in a same physical space, and the medical worker may control the medical device to acquire the medical image for the patient. However, in areas with underdeveloped medical resources and remote areas, there may be a lack of medical workers, which limits the progress of medical operation. In addition, for a patient with an infectious disease, the medical worker and the patient being in the same physical space may increase the risk of infection for the medical worker. Accordingly, the embodiments of the present disclosure provides a medical method in which twin data associated with the object may be transmitted to a control terminal device corresponding to the medical worker to enable the medical worker in a first space to control the medical device in a second space, so as to realize the medical operation of the object in the space where the medical device is located. In this way, when the object (e.g., the patient) and the medical worker are in different physical spaces, the medical operation can also be realized, which not only breaks the distance limit of medical operation, but also can perform a remote medical operation on objects in the areas with underdeveloped medical conditions or objects with the infectious disease, thereby making medical operation more convenient and faster.

FIG. 11 is a flowchart illustrating an exemplary process for remote medical operation method according to some embodiments of the present disclosure. In some embodiments, process 1100 may be executed by the medical system 100. For example, the process 1100 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 1100. The operations of the illustrated process presented below are intended to be illustrative. In some embodiments, the process 1100 may be accomplished with one or more additional operations not described and/or without one or more of the operations discussed. Additionally, the order of the operations of process 1100 illustrated in FIG. 11 and described below is not intended to be limiting.

In 1110, the processing device 120 (e.g., the processor 210) may obtain twin data associated with an object from an operation terminal device.

As shown in FIG. 1, the operation terminal device may be a terminal device used by the object, which may be located in a physical space different from a physical space where a control terminal device is located used by a medical worker. Accordingly, a user (i.e., the medical worker) of the control terminal device and a user (i.e., the object) of the operation terminal device may be also located in different physical spaces. For example, the control terminal device may be located in a first space where the medical worker is located, and the operation terminal device may be located in a second space where the object is located. Both the first space and the second space may be real physical spaces, and a geographic position of the first space may be different from the geographic position of the second space.

In some embodiments, the twin data associated with the object may include twin data corresponding to the object and/or twin data corresponding to a medical device corresponding to the operation terminal device.

The twin data corresponding to the object refers to virtual information (e.g., a virtual image of the object) associated with the object. In some embodiments, the twin data corresponding to the object may be displayed in a twin space (e.g., a virtual space viewed through the display device 140). In some embodiments, the twin data corresponding to the object may be a stereoscopic virtual image (e.g., a three-dimensional stereoscopic projection).

In some embodiments, the twin data corresponding to the object may be generated in real time or in advance. For example, the processing device 120 may generate the twin data corresponding to the object in real time based on target information of the object. As another example, the processing device 120 may read the twin data corresponding to the object from a storage device (e.g., the storage device 150) generated in advance based on the target information of the object.

In some embodiments, the processing device 120 may generate the twin data corresponding to the object based on the target information of the object. The target information may include personal information (e.g., age, height, weight, medical history, gender), imaging requirement information (e.g., an organ structure of a scanning site), etc. For example, the processing device 120 may generate the twin data corresponding to the object through simulation based on the personal information and image requirement information of the object.

In some embodiments, the processing device 120 may generate the twin data corresponding to the object based on image data (e.g., multimedia data, image data, audio data, video data) associated with the object. In some embodiments, the image data associated with the object may be acquired by the acquisition device and transmitted to the operation terminal device, and the operation terminal device may then transmit the image data to the processing device 120; or the acquisition device may directly transmit the image data to the processing device 120. In some embodiments, a field of view of the acquisition device may cover all or part of the second space. For example, the field of view of the acquisition device may completely cover the medical device in the second space and an entrance of the second space.

In some embodiments, the processing device 120 may determine a pose (e.g., a posture, a position point, a position area) of the object, a positional relationship (e.g., a distance, an angle) between the object and the medical device, etc., and then generate the twin data corresponding to the object. Merely by way of example, the processing device 120 may identify the position area of the object in the second space and the posture of the object in the second space by processing the image data. Further, the processing device 120 may determine the positional relationship between the object and the medical device according to the position area of the object in the second space and an area where a medical bed (e.g., the medical bed 116) is located and generate the twin data corresponding to the object according to the positional relationship and the posture of the object.

The twin data corresponding to a medical device corresponding to the operation terminal device refers to virtual information (e.g., a virtual image of the medical device) associated with the medical device. In some embodiments, the twin data corresponding to the medical device may be displayed in the twin space. In some embodiments, the twin data corresponding to the medical device may be a stereoscopic virtual projection (e.g., a three-dimensional stereoscopic projection of the medical device) in the twin space.

In some embodiments, the processing device 120 may generate the twin data corresponding to the medical device based on real-time state information of the medical device. For example, the processing device 120 may receive the state information of the medical device sent by the operation terminal device in real time, generate rendering data of the medical device based on the state information of the medical device, and determine the rendering data as the twin data corresponding to the medical device.

In some embodiments, the state information of the medical device may reflect system parameter information of the medical device. Merely by way of example, when the medical device is an MRI device, the state information of the medical device may include coil information of the MRI device, inspection layer thickness information, interlayer distance information, slice direction information, radio frequency pulse sequence information, imaging sequence information, image acquisition bed height information, or the like, or any combination thereof. As another example, when the medical device is a CT device, the state information of the medical device may include scanning type information, exposure information, layer thickness information, layer distance information, field of view information, image acquisition posture information, image acquisition bed height information, or the like, or any combination thereof. In some embodiments, the state information of the medical device may be uploaded to the processing device 120 by the operation terminal device or the medical device itself, which is not limited in the present disclosure.

In 1120, the processing device 120 (e.g., the processor 210) may transmit the twin data associated with the object to a control terminal device.

As mentioned above, the control terminal device refers to a terminal device used by medical worker. In some embodiments, after receiving the twin data, the control terminal device may display the twin data associated with the object in the twin space. In some embodiments, the control terminal device may direct a first display device corresponding to the control terminal device to display the twin data associated with the object in the twin space. In some embodiments, the control terminal device and the first display device corresponding to the control terminal device may be different modules of the same device. For example, the control terminal device and the first display device corresponding to the control terminal device may be a control module and a display module of the VR wearable device, respectively. In some embodiments, the control terminal device and the first display device corresponding to the control terminal device may be separate devices. For example, the control terminal device and the first display device corresponding to the control terminal device may be a first control device and the first display device (e.g., a first wearable device or an optical projection device) communicatively connected with each other.

Merely by way of example, after the processing device 120 transmits the twin data corresponding to the object to the control terminal device, the first wearable device worn by the medical worker may obtain the twin data corresponding to the object from the control terminal device and display the twin data corresponding to the object, and the medical worker may view the twin data corresponding to the object in the twin space through the first wearable device to obtain the position and/or posture of the object.

As another example, after the processing device 120 transmits the twin data corresponding to the medical device to the control terminal device, the control module of the control terminal device may control the display module to display the twin data corresponding to the medical device, and the medical worker may view the twin data corresponding to the medical device in the twin space through the display module to obtain terminal information of the medical device and/or a relative position between the medical device and the object.

In some embodiments, the processing device 120 may also transmit the twin data corresponding to the object to the operation terminal device. For example, after generating the twin data corresponding to the object based on the target information or image data of the object, the processing device 120 may transmit the twin data corresponding to the object to the operation terminal device, then a display device (also referred to as a second display device) corresponding to the operation terminal device may display the target information of the object based on the twin data corresponding to the object, and the object may view the target information or image data in the twin space through the second display device.

In some embodiments, after displaying the twin data (e.g., the twin data corresponding to the object, the twin data corresponding to the medical device) in the twin space, the control terminal device may determine guidance data which may be used to guide the object to perform a preparation operation for a medical operation.

In some embodiments, the guidance data may include a picture, a video, an audios, etc. used to guide the object to perform the preparation operation. In some embodiments, the guidance data may include position guidance data, posture guidance data, or the like, or any combination thereof.

The position guidance data refers to guidance data that guides the object to a target area and/or a target position. For example, the position guidance data may include position guidance information in the form of a video, which may be used to guide the object to move to the target area and/or the target position. In some embodiments, the target area and/or the target position may include an area and/or a position where the medical operation is to be performed, for example, an area and/or a position where the medical bed 116 is located, an area and/or a position where the medical device 110 is located, etc.

The posture guidance data refers to guidance data that guides the object to maintain a target posture or change a posture. For example, the posture guidance data may include posture guidance information in the form of a picture sequence, which may be used to guide the object to maintain the target posture. In some embodiments, the target posture may be a posture (e.g., lying supine, lying prone, side lying, sitting) that needs to be maintained during the medical operation. For example, if a medical image of a lateral position of the lumbar spine of the object needs to be acquired, a side-lying posture may need to be maintained; if a medical image of a normal position of the lumbar spine of the object needs to be acquired, a lying posture may need to be maintained. As another example, when image data of the object is to be acquired, the object may need to remain in a static state.

In some embodiments, the guidance data may further include process guidance data. The process guide data refers to guidance data that guides a sequence of preparation operations to be performed by the object. For example, the process guidance data may include sequence guidance information (e.g., changing clothes→moving to the target area→maintaining the target posture) in the form of video that demonstrates the preparation operations that the object needs to perform before the medical image is acquired.

In some embodiments, the guidance data may be generated in advance or in real time.

In some embodiments, the control terminal device may determine the guidance data based on a state (e.g., a position and/or a posture of the object, a position of the medical device, a relative positional relationship between the object and the medical device) of the medical device and/or the object.

In some embodiments, the control terminal device may generate the position guidance data when the object is not in the target area and/or the target position. In some embodiments, the control terminal device may determine whether the object is located in the target area and/or the target position based on the relative positional relationship between the object and the medical device. For example, the control terminal device may determine the relative positional relationship between the object and the medical device based on the position of the object and the position of the medical device, thereby determining whether the object is located in the target area and/or the target position and generating the position guidance data when the object is not in the target area and/or the target position.

In some embodiments, the control terminal device may generate the posture guidance data when the object is located in the target area and/or the target position but is not in the target posture. In some embodiments, the control terminal device may determine whether the object is in the target posture based on the posture of the object. For example, the control terminal device may determine the relative positional relationship between the object and the medical device based on the position of the object and the position of the medical device, determine whether the object is located in the target area and/or the target position according to the relative positional relationship, determine whether the object is in the target posture according to the posture of the object, and generate the posture guidance data when the object is not in the target posture. For example, if the target posture is a side-lying posture, the object is on a medical bed, but the posture of the object is a lying posture, it may mean that the object is not in the target posture, and the control terminal device may generate the posture guidance data corresponding to the side-lying posture and transmit the posture guidance data to the processing device 120.

In some embodiments, the control terminal device may display the twin data associated with the object in the twin space and guide the medical worker to determine the guidance data according to the displayed twin data associated with the object. The data in the twin space may reflect the state information of the medical device and the object in the second space, and medical worker can obtain the state (e.g., the position of the object, the posture of the object, the position of the medical device, the relative positional relationship between the object and the medical device) of the object and/or the medical device in the second space based on the twin data displayed in the twin space, so as to determine the guidance data. For example, the medical worker may determine a scanning site of the object that needs to be scanned for a medical scanning and the target posture when the medical image is acquired by viewing the twin data associated with the object.

In some embodiments, the control terminal device may retrieve the guidance data generated in advance or guidance data generated in real time from a storage device (e.g., the storage device 150) based on an operation (e.g., a gesture operation, a voice operation, an eye movement, a brain wave operation, a touch operation) of the medical worker.

Merely by way of example, the control terminal device may issue a guidance data determination instruction to the medical worker by displaying the twin data in the twin space. After viewing the twin data associated with the object in the twin space and determining that the object is not in the target area and/or the target position (e.g., the object is not in an area where the medical bed 116 is located), the medical worker may select the corresponding position guidance information on the control terminal device through the touch operation, and the control terminal device may read the corresponding position guidance data from the storage device 150 based on a touch instruction.

As another example, after the control terminal device issues the guidance data determination instruction to the medical worker, the medical worker may view the twin data associated with the object in the twin space. When the object is in the target area and/or the target position (e.g., the object is in the area where the medical bed 116 is located), and the posture of the object is not the target posture, the medical worker may initiate a call instruction of the posture guidance data using the voice operation, and the control terminal device may generate the posture guidance data in real time based on the call instruction. For example, if the target posture is a side-lying posture, the medical worker finds that the object is on the medical bed (i.e., the object is in the target area) through the twin data corresponding to the object displayed in the twin space, but the posture of the object is a lying posture, it may mean that the object is not in the target posture, and the medical worker may initiate the call instruction of the posture guidance data through the voice operation, the touch operation, the gesture operation, etc.

In 1130, the processing device 120 (e.g., the processor 210) may obtain guidance data from the control terminal device.

In some embodiments, after determining the guidance data, the control terminal device may transmit the guidance data to the processing device 120. In some embodiments, the control terminal device may transmit the guidance data to the processing device 120 based on a guidance transmitting instruction. In some embodiments, the control terminal device may generate the guidance transmitting instruction based on a gesture operation (e.g., a gesture action performed by an operation handle), a voice operation, a touch operation (e.g., an operation such as clicking and sliding on a display screen or keyboard), etc.

In 1140, the processing device 120 (e.g., the processor 210) may direct a display device associated with the operation terminal device to present the guidance data.

In some embodiments, the processing device 120 may direct the display device (e.g., the second display device) associated with the operation terminal device to present the guidance data to guide the object to perform the preparation operation.

Similar to the control terminal device, the operation terminal device and the second display device corresponding to the operation terminal device may be different modules of the same device, or two different devices connected through communication. For example, the operation terminal device may be a second control device, and the second display device may be an optical projection device, an electronic display screen, a second wearable device worn by the object, etc. in the same space (e.g., the second space) as the second control device. As another example, the operation terminal device may be the control module of the VR wearable device, and the second display device may be the display screen of the VR wearable device.

In some embodiments, after receiving the guidance data, the processing device 120 may process (e.g., render) the guidance data to obtain target guidance data suitable for being displayed on the second display device. Further, the processing device 120 may transmit the target guidance data to the second display device, so that the second display device may display guidance information in the twin space according to the target guidance data. Merely by way of example, the second wearable device (e.g., the VR wearable device, the projection device) worn by the object may display the position guidance information and/or the posture guidance information in the twin space based on the target guidance data sent by the processing device 120. The object may view the position guidance information and/or the posture guidance information in the twin space through the second wearable device, so as to perform the preparation operation according to the position guidance information and/or the posture guidance information.

In some embodiments, before obtaining the twin data associated with the object, the processing device 120 may transmit twin data associated with the medical worker to the operation terminal device. For example, the processing device 120 may obtain the twin data associated with the medical worker in response to receiving a login request sent by the control terminal device; and transmit the twin data associated with the medical worker to the operation terminal device. After receiving the twin data associated with the medical worker, the operation terminal device may display the twin data (e.g., a virtual image of the medical worker) corresponding to the medical worker in the twin space.

In some embodiments, the login request may be initiated by the medical worker. For example, when the control terminal device and the first display device are different modules of the same device, the medical worker may initiate the login request through the control terminal device, and the control terminal device may transmit the login request to the processing device 120 in response to a login control being triggered. As another example, when the control terminal device and the first display device are different devices (e.g., a first control device and a first display device communicated with each other), the medical worker may initiate the login request through the first display device, an operation component (e.g., the operation handle) of the first display device may obtain login gesture data of the medical worker and transmit the login gesture data to the first control device, and the first control device may parse the login gesture data to obtain the login request and transmit the login request to the processing device 120.

In some embodiments, the twin data associated with the medical worker may be generated in real time or in advance. For example, after receiving the login request, the processing device 120 may obtain the twin data associated with the medical worker generated in advance from the storage device 150. As another example, after receiving the login request, the processing device 120 may generate the twin data associated with the medical worker in real time based on personal information of the medical worker.

In some embodiments, the processing device 120 may obtain twin data associated with an operation page (e.g., a control page used to control the medical device) in response to receiving the login request sent by the control terminal device; and transmit the twin data associated with the operation page to the control terminal device. After receiving the twin data associated with the operation page, the control terminal device may display the twin data associated with the operation page in the twin space. In some embodiments, the operation page may include configuring an operational parameter (e.g., various parameters such as a scanning mode, a scanning sequence, etc. of the medical device), controlling the medical device to acquire the medical image, etc. Accordingly, the twin data associated with the operation page may include virtual information (e.g., a virtual projection of the control page) corresponding to the control page.

In some embodiments, the twin data associated with the operation page may be generated in advance or in real time. For example, the processing device 120 may obtain the twin data associated with the operation page generated in advance from the storage device 150 based on a model of the medical device. As another example, the processing device 120 may generate the twin data associated with the operation page in real time based on performance, a parameter type, the model, etc. of the medical device.

In some embodiments, the twin data associated with the operation page displayed in the twin space may be consistent with the control page of the medical device displayed on the operation terminal device. For example, a layout and/or contents of the control page displayed after the medical device is started may be consistent with a layout and contents of the twin data associated with the operation page twin displayed in the twin space by the control terminal device. The medical worker may view the twin data associated with the operation page through the display device (e.g., the first display device) and perform a touch operation, so as to realize setting of the control page of the medical device (e.g., setting of the operational parameter).

In some embodiments, the twin data associated with the medical worker and/or the twin data associated with the operation page may be pre-stored in a storage device (e.g., the storage device 150, the memory 220).

It should be noted that the above description regarding the process 600 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 600 may be accomplished with one or more additional operations not described and/or without one or more of the operations discussed above.

FIG. 12 is a flowchart illustrating an exemplary process for remote medical operation method according to other embodiments of the present disclosure. In some embodiments, process 1100 may be executed by the medical system 100. For example, the process 1200 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 1200. The operations of the illustrated process presented below are intended to be illustrative. In some embodiments, the process 1200 may be accomplished with one or more additional operations not described and/or without one or more of the operations discussed. Additionally, the order of the operations of process 1200 illustrated in FIG. 12 and described below is not intended to be limiting.

In 1210, the processing device 120 (e.g., the processor 210) may obtain twin data associated with an object from an operation terminal device. More descriptions may be found in operation 1110.

In 1220, the processing device 120 (e.g., the processor 210) may transmit the twin data associated with the object to a control terminal device. More descriptions may be found in operation 1120.

In 1230, the processing device 120 (e.g., the processor 210) may obtain guidance data from the control terminal device, wherein the guidance data is determined based on the twin data associated with the object. More descriptions may be found in operation 1130.

In 1240, the processing device 120 (e.g., the processor 210) may direct a display device associated with the operation terminal device to present the guidance data. More descriptions may be found in operation 1140.

In 1250, the processing device 120 (e.g., the processor 210) may obtain a control instruction from the control terminal device.

As mentioned above, the control instruction may include a control instruction associated with the operational parameter of the medical operation. In some embodiments, the control instruction associated with the operational parameter may include an instruction for setting and/or adjusting the operational parameter, for example, an instruction for setting and/or adjusting a scanning time, a scanning sequence, a scanning site, a scanning speed, a scanning intensity, a system parameter of medical device. In some embodiments, the control instruction associated with the operational parameter may include a target value/target mode of the operational parameter, and/or an adjustment mode (e.g., increasing, decreasing, remaining unchanged, a magnitude increased or decreased) of the operational parameter.

In some embodiments, as described in connection with FIG. 11, the control instruction may include an adjustment instruction associated with the state information of the medical device. For example, when the medical device is an MRI device, the control instruction may include adjusting ventilation of the MRI device, coil information (e.g., a coil type) of the MRI device, radio frequency pulse sequence information (e.g., an amplitude and period of the pulse sequence), image acquisition bed height information (e.g., a height value of the medical bed 116), etc. As another example, when the medical device is a CT device, the control instruction may include adjusting ventilation of the CT device, exposure information of the CT device, image acquisition posture information, a height of the image acquisition bed, etc.

In some embodiments, the control instruction may also include an image acquisition instruction. The image acquisition instruction may be used to direct the medical device to acquire the medical image of the object. For example, the medical device may emit X-rays to the object based on the image acquisition instruction and receive X-rays through a detector, so as to obtain the medical image of the object.

In some embodiments, the image acquisition instruction may be generated after the setting or adjustment of the operational parameter is completed. In some embodiments, the image acquisition instruction may be generated directly. For example, when the operational parameter does not need to be set or adjusted (e.g., the state information of the medical device does not need to be adjusted), the control terminal device may directly transmit the image acquisition instruction.

In some embodiments, the control terminal device may determine the control instruction based on a gesture operation (e.g., a gesture action performed by an operation handle), a voice operation, a touch operation (e.g., an operation such as clicking and sliding on a display screen or keyboard), etc. of the medical worker and transmit the control instruction to the processing device 120. In some embodiments, the control terminal device may determine operation content(s) triggered by the medical worker through the twin data corresponding to the operation page and determine the control instruction based on the operation content(s). For example, the control terminal device may transmit the adjustment instruction to the processing device 120 in response to an adjustment operation (e.g., a specific gesture operation performed by the operation handle) triggered by the medical worker through the twin data corresponding to the operation page. As another example, the control terminal device may transmit the image acquisition instruction operation to the processing device 120 in response to an image acquisition operation (e.g., clicking on the display screen) triggered by the medical worker through the twin data corresponding to the operation page.

In some embodiments, the control terminal device may obtain operation data (e.g., an operation gesture, a voice content, a touch area/point) of the medical worker through an input device (e.g., a keyboard, an operation handle, a mouse, a touch screen) and obtain the control instruction by analyzing the operation data. For example, the operation handle of a first display device (e.g., a first wearable device) may obtain posture data of the medical worker and determine gesture operation data according to a correspondence between the posture data and the operation gesture. Merely by way of example, the gesture operation data corresponding to leaning the body to the left is sliding left, the gesture operation data corresponding to leaning the body to the right is sliding right, the gesture operation data corresponding to leaning the body forward is sliding up, the gesture operation data corresponding to leaning the body backward is sliding down, etc.

In some embodiments, after the object completes an preparation operation based on the guidance data, the processing device 120 may obtain the twin data associated with the object again and transmit the twin data to the control terminal device, so that the medical worker can determine whether the object has completed the preparation operation. When determining that the object has completed the preparation operation, the medical worker may transmit the control instruction through the control terminal device.

In 1260, the processing device 120 (e.g., the processor 210) may transmit the control instruction to the operation terminal device to control a medical device corresponding to the operation terminal device to control a medical operation. In some embodiments, after receiving the control instruction sent by the processing device 120, the operation terminal device may transmit the control instruction to the medical device, and the medical device may perform a corresponding operation (e.g., configuring the operational parameter, acquiring the medical image) according to the control instruction. In some embodiments, after receiving the control instruction sent by the processing device 120, the operation terminal device may directly control the medical device to perform the corresponding operation according to the control instruction. For example, the processing device 120 may transmit the adjustment instruction associated with the state information of the medical device to the operation terminal device, and the operation terminal device may control the medical device to adjust the state information based on the adjustment instruction. As another example, the medical worker may transmit the image acquisition instruction through the control terminal device, the processing device 120 may transmit the image acquisition instruction to the operation terminal device, the operation terminal device may forward the image acquisition instruction to the medical device, and the medical device may perform an imaging operation on the object based on the image acquisition instruction so as to obtain the medical image of the object.

In some embodiments, after the medical device completes the adjustment of the state information of the medical device based on the control instruction, the processing device 120 may obtain updated twin data corresponding to the medical device and transmit the updated twin data corresponding to the medical device to the control terminal device.

In some embodiments, the processing device 120 may directly transmit the control instruction to the medical device.

For example, when the medical device is the MRI device and the control instruction is used to adjust the height information of the image acquisition bed, the processing device 120 may transmit the control instruction to the MRI device, and the MRI device may adjust the height of the image acquisition bed of the MRI device according to the control instruction. After the adjustment is completed, the MRI device may transmit a current height of the image acquisition bed to the processing device 120, and the processing device 120 may direct the display device (e.g., the first display device, the second display device) to update a height of an image acquisition bed in the twin space.

As another example, when the medical device is the CT device, and the control instruction is used to adjust the image acquisition posture information, the processing device 120 may transmit the control instruction to the CT device, and the CT device may adjust the image acquisition posture of the CT device from a first posture is to a second posture. After the adjustment is completed, the CT device may transmit a current image acquisition posture to the processing device 120, and the processing device 120 may direct the display device (e.g., the first display device, the second display device) to adjust an image acquisition posture in the twin space from the first posture to the second posture.

In some embodiments, after the imaging operation is completed, the medical device may transmit the medical image of the object to the processing device 120.

In some embodiments, the processing device 120 may transmit the medical image to the control terminal device, so that the medical worker can view the medical image through the control terminal device. For example, the processing device 120 may determine image twin data (e.g., a virtual medical image) based on the medical image and transmit the image twin data to the control terminal device. The control terminal device may control the first display device to display the image twin data, and the medical worker can view the image twin data in the twin space through the first display device (e.g., the VR wearable device). As another example, the processing device 120 may transmit the medical image to the control terminal device through the network 160, and the control terminal device may display the medical image in a display interface to output to the medical worker.

In some embodiments, the processing device 120 may transmit the medical image to the operation terminal device. For example, the processing device 120 may determine the image twin data based on the medical image and transmit the image twin data to the operation terminal device. The operation terminal device may display the image twin data in the twin space, and the object can view the image twin data in the twin space through the second display device (e.g., a second wearable device). As another example, the processing device 120 may transmit the medical image to the operation terminal device through the network 160, and the operation terminal device may display the medical image in a display interface to output to the object.

According to the embodiments of the present disclosure, in the first space where the control terminal device is located, the medical worker can control the medical device located in the second space through the interaction between the control terminal device, the operation terminal device, and the processing device, so as to realize the remote control of a medical operation in the second space and break the geographic position limitation.

In some embodiments, the processing device 120 may receive dialog data sent by the control terminal device and/or the operation terminal device, generate twin data associated with the dialog data, and transmit the twin data associated with the dialog data to the operation terminal device and/or the control terminal device. The twin data associated with the dialog refers to a copied audio or text of a dialog. For example, the processing device 120 may receive first dialog data of the control terminal device, generate first twin data associated with the dialog based on the first dialog data, and transmit the first twin data associated with the dialog to the operation terminal device and/or the control terminal device. The operate terminal device and/or the control terminal device may play first dialog information based on the first twin data associated with the dialog. As another example, the processing device 120 may receive second dialog data sent by the operation terminal device, generate second twin data associated with the dialog based on the second dialog data, and transmit the second twin data associated with the dialog to the operation terminal device and/or the control terminal device. The operate terminal device and/or the control terminal device may play second dialog information based on the second twin data associated with the dialog.

In some embodiments, the first dialog data may be first voice data of the medical worker, and the second dialog data may be second voice data of the object. The control terminal device may record the first voice data and transmit the first voice data to the processing device 120. The operation terminal device may record the second voice data and transmit the second voice data to the processing device 120. The control terminal device may play the first dialog information and the second dialog information in the twin space, and the operation terminal device may play the first dialog information and the second dialog information in the twin space, so that the medical worker and the object can hear the first dialog information and second dialog information.

According to the embodiments of the present disclosure, when the object and the medical worker are in different physical spaces, through the twin space, the object and the medical worker can have a real-time dialog through the first dialog data and the second dialog data, which can provide an immersive scene of communicating in the same physical space for the object and the medical worker.

In some embodiments, the processing device 120 may receive error message sent by the operation terminal device and transmit the error message to the control terminal device. In some embodiments, the processing device 120 may query a solution corresponding to the error message and transmit the solution to the operation terminal device. For example, when the operation terminal device or the medical device fails, the operation terminal device may transmit the error message to the processing device 120. If the processing device 120 is preset with a solution corresponding to the error message, the solution corresponding to the error message may be sent to the operation terminal device. If the solution corresponding to the error message is not preset, the processing device 120 may transmit the error message to the control terminal device, and the control terminal device may transmit an error prompt corresponding to the error message, so that medical worker may notice that the operation terminal device or the medical device has failed, the medical worker may learn details of the fault based on the error message and transmit the corresponding solution to the processing device 120 through the control terminal device, and the processing device 120 may transmit the solution corresponding to the error message to the operation terminal device.

It should be noted that the above description regarding the process 1200 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 1200 may be accomplished with one or more additional operations not described and/or without one or more of the operations discussed above. In some embodiments, the control terminal device may be local and the operation terminal device may be remote. In some embodiments, the operation terminal device may be local and the control terminal device may be remote.

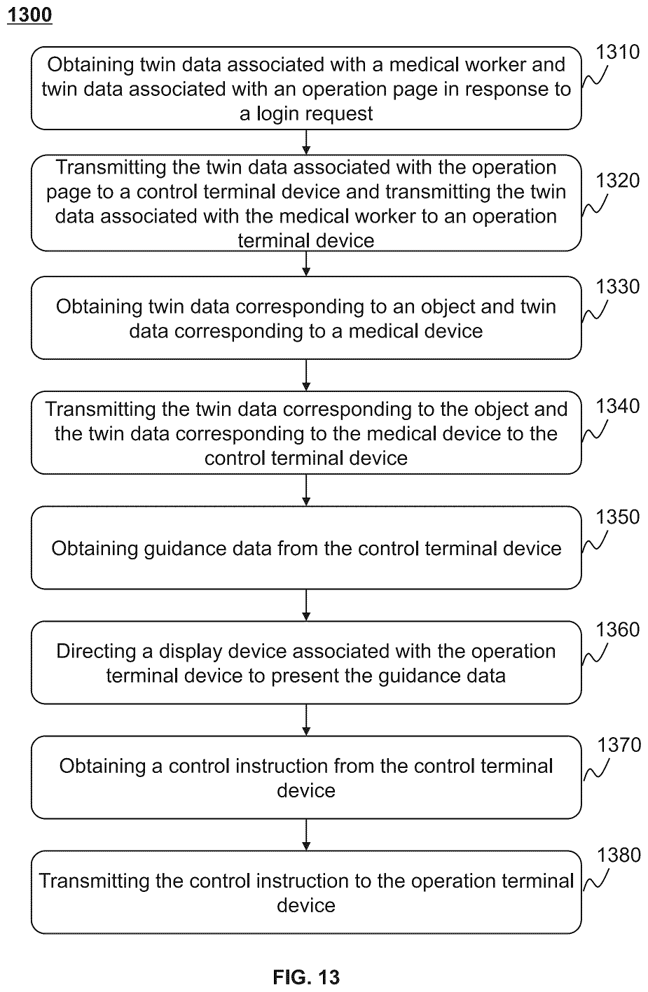

FIG. 13 is a flowchart illustrating an exemplary process for remote medical operation according to other embodiments of the present disclosure. In some embodiments, process 1300 may be executed by the medical system 100. For example, the process 1300 may be implemented as a set of instructions stored in the storage device (e.g., the storage device 150). In some embodiments, the processing device 120 (e.g., the processor 210 of the computing device 200 and/or one or more modules illustrated in FIG. 3) may execute the set of instructions and may accordingly be directed to perform the process 1300. The operations of the illustrated process presented below are intended to be illustrative. In some embodiments, the process 1300 may be accomplished with one or more additional operations not described and/or without one or more of the operations discussed. Additionally, the order of the operations of process 1300 illustrated in FIG. 13 and described below is not intended to be limiting.

In 1310, the processing device 120 (e.g., the processor 210) may obtain twin data associated with a medical worker and twin data associated with an operation page in response to a login request.

As described in connection with above, the medical worker may initiate the login request (e.g., logging in a user account, performing a login gesture) through a control terminal device. In some embodiments, the processing device 120 may obtain the twin data associated with the medical worker and the twin data associated with the operation page from a storage device (e.g., the storage device 150) in response to the login request received from the control terminal device. More descriptions may be found in FIG. 11, which will not be repeated herein.

In 1320, the processing device 120 (e.g., the processor 210) may transmit the twin data associated with the operation page to the control terminal device and transmit the twin data associated with the medical worker to the operation terminal device.

In some embodiments, the processing device 120 may transmit the twin data associated with the medical worker to the operation terminal device, so that the operation terminal device may display the twin data associated with the medical worker. In some embodiments, the processing device 120 may transmit the twin data associated with the operation page to the control terminal device, so that a first display device corresponding to the control terminal device may display the twin data associated with the operation page in a twin space. More descriptions may be found in FIG. 11, which will not be repeated herein.

In 1330, the processing device 120 (e.g., the processor 210) may obtain twin data corresponding to an object and twin data corresponding to a medical device.

More descriptions may be found in the operation 1110, which will not be repeated herein.

In 1340, the processing device 120 (e.g., the processor 210) may transmit the twin data corresponding to the object and the twin data corresponding to the medical device to the control terminal device.

More descriptions may be found in the operation 1120, which will not be repeated herein.

In 1350, the processing device 120 (e.g., the processor 210) may obtain guidance data from the control terminal device.

More descriptions may be found in the operation 1130, which will not be repeated herein.

In 1360, the processing device 120 (e.g., the processor 210) may direct a display device associated with the operation terminal device to present the guidance data.

More descriptions may be found in the operation 1140, which will not be repeated herein.

In 1370, the processing device 120 (e.g., the processor 210) may obtain a control instruction from the control terminal device.

More descriptions may be found in the operation 1250, which will not be repeated herein.

In 1380, the processing device 120 (e.g., the processor 210) may transmit the control instruction to the operation terminal device.

More descriptions may be found in the operation 1260, which will not be repeated herein.

It should be noted that the above description regarding the process 1300 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

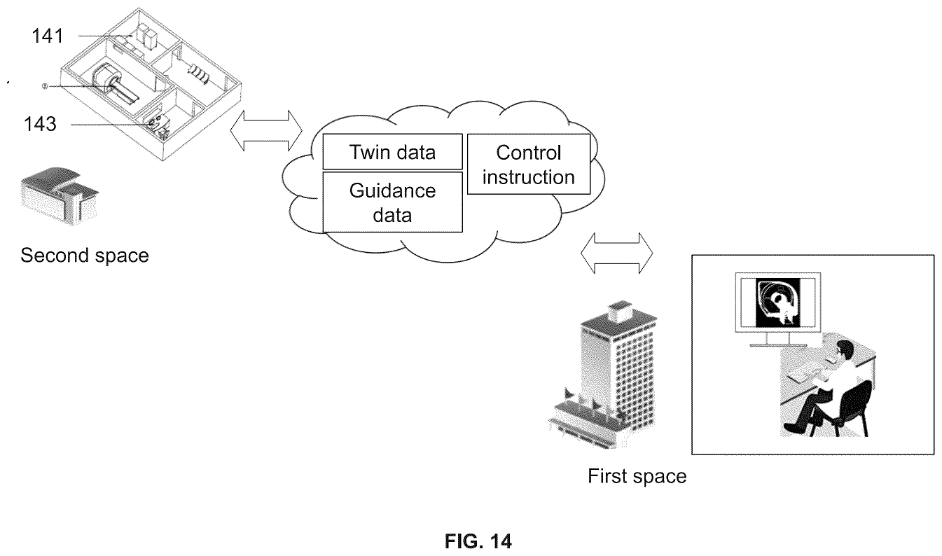

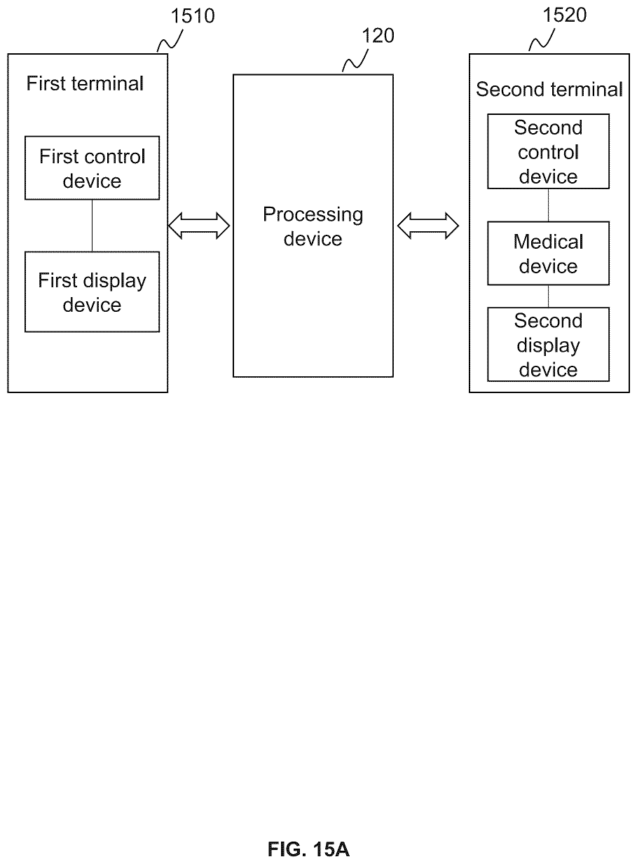

FIG. 14 is a schematic diagram illustrating an exemplary scene of a first space and a second space according to some embodiments of the present disclosure.

In some embodiments, the first space may be a space corresponding to a specialist hospital, and the second space may be a space corresponding to a primary hospital. A medical worker may be an expert who works in the specialist hospital, and an object may be an object who is treated in the primary hospital.

As shown in FIG. 14, in some embodiments, the second space may include a power amplifier device room 141, a scanning room 142, and an operation room 143. The scanning room 142 may be used to place a medical device, and the operation room 143 may be used to place an operation terminal device. The power amplifier device room 141 and the scanning room 143 may be used to assist the medical device to complete a scanning process to obtain an medical image of the object.

In some embodiments, the first space may include a control terminal device. The control terminal device may remotely obtain (e.g., from the processing device 120) information (e.g., twin data corresponding to the object, twin data corresponding to the medical device) of the power amplifier device room 141, the scanning room 142, and the operation room 143 through a cloud network (e.g., the network 160). The operation terminal device in the second space may obtain (e.g., from the processing device 120) guidance data sent by the medical worker through the cloud network. The control terminal device may also transmit a control instruction to the medical device in the second space through the cloud network, so that the medical worker can control the medical device and acquire the medical image of the object remotely, or the medical worker can guide a scanning technician in the primary hospital to acquire the medical image of the object who is treated in the primary hospital.

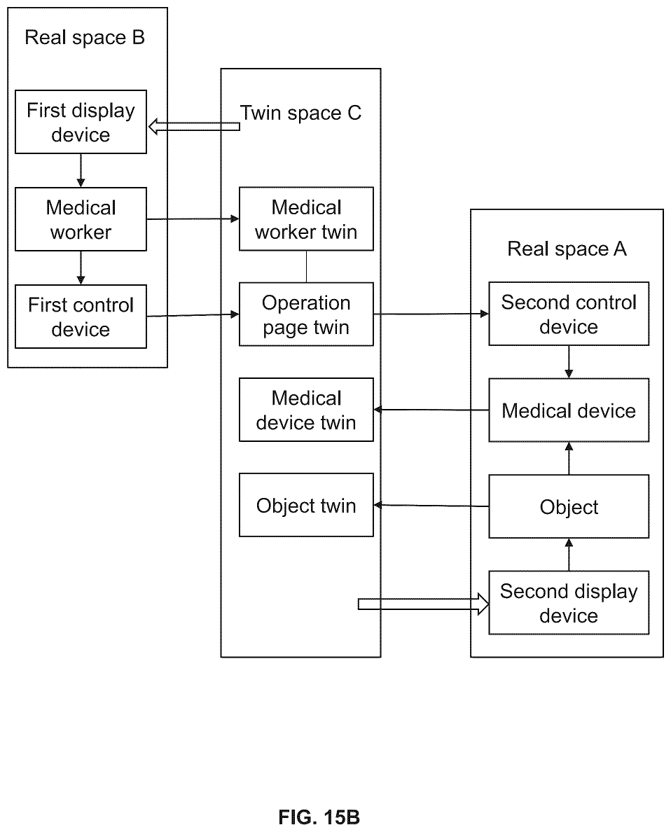

FIGS. 15A and 15B are schematic diagrams illustrating exemplary medical scenes according to some embodiments of the present disclosure.

For the convenience of illustration, an example in which an operation terminal device and a second display device are different devices, and a control terminal device and a first display device are different devices is provided below, it should be noted that the operation terminal device and the second display device may be implemented via a same device, and the control terminal device and the first display device may be implemented via a same device.

As illustrated in FIG. 15A, the control terminal device may correspond to the first control device, and the operation terminal device may correspond to the second control device. A combination of the first control device and the first display device in a first space may be referred to as a first terminal 1510, and a combination of the second control device, a medical device, and the second display device in a second space may be referred to as a second terminal 1520.

In some embodiments, before a scanning process, the processing device 120 may transmit first twin data (e.g., twin data corresponding to an object, twin data corresponding to the medical device) corresponding to the second terminal 1520 to the first terminal 1510. The first terminal 1510 may obtain the first twin data corresponding to the second terminal 1520 from the processing device 120 and determine guidance data and/or a control instruction based on the first twin data, and further transmit the guidance data (e.g., position guidance data, posture guidance data) to the processing device 120. The processing device 120 may further transmit the guidance data and/or the control instruction to the second terminal 1520. In some embodiments, the processing device 120 may also transmit twin data (e.g., twin data corresponding to the medical worker) corresponding to the first terminal 1510 to the second terminal 1520.

As illustrated in FIG. 15B, the medical worker is in a real space B (i.e., the first space), and the object is in a real space A (i.e., the second space). The real space B may include the first display device and the first control device. The medical worker can issue the guidance data, the control instruction, and/or dialog data through the first control device. The real space A may include the medical device, the second control device, and the second display device. The object can receive the guidance data, the control instruction, and/or the dialog data through the second control device and/or the second display device. For example, the medical worker in the real space B may initiate the login request by means of account login, then the processing device 120 may direct the first display device to display twin data (also can be referred to as “operation page twin”) corresponding to the operation page, twin data (also can be referred to as “object twin”) corresponding to the object, and twin data (also can be referred to as “medical device twin”) corresponding to the medical device in response to the login request, and direct the second display device in the real space A to display twin data (also can be referred to as “medical worker twin”) corresponding to the medical worker. The medical worker in the real space B can view the object twin, the operation page twin, and the medical device twin in a twin space C through the first display device and transmit the guidance data and the control instruction through the first control device. After obtaining the guidance data and/or the control instruction, the processing device 120 may direct the second display device in the real space A to display the guidance data and the control instruction. The object in the real space A can view the medical worker twin, the guidance data, and the control instruction in the twin space C through the second display device in the real space A.

The embodiments of the present disclosure also provide a computer device, including a storage storing a computer program and a processor. The processor may implement the operations in the embodiments of the present disclosure when executing the computer program.

The embodiments of the present disclosure also provide a computer-readable storage medium storing a computer program. When the computer program is executed by a processor, the operations in the embodiments of the present disclosure may be implemented.

The embodiments of the present disclosure also provide a computer program product including a computer program. When the computer program is executed by a processor, the operations in the embodiments of the present disclosure may be implemented.

It should be noted that user information (including but not limited to user device information, user personal information) and data (including but not limited to data used for analysis, storage, display) involved in the present disclosure are all information and data authorized by a user or fully authorized by all parties.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, some features, structures, or features in the present disclosure of one or more embodiments may be appropriately combined.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A system, comprising:
at least one storage medium including a set of instructions; and
at least one processor in communication with the at least one storage medium, wherein when executing the set of instructions, the at least one processor is directed to cause the system to perform operations including:
obtaining a call instruction of virtual caregiver information, wherein the virtual caregiver information includes accompany information associated with a caregiver, the caregiver is related to an object, and the accompany information includes at least one of visual information, auditory information, or tactile information; and
directing a display device to display the virtual caregiver information based on the call instruction.

2. The system of claim 1, wherein before the call instruction of the virtual caregiver information is obtained, the at least one processor is further directed to cause the system to perform operations including:
directing the display device to display a scanning protocol associated with the object based on a call instruction of the scanning protocol.

3. The system of claim 1, wherein the at least one processor is further directed to cause the system to perform operations including:
receiving a feedback from the object; and
adjusting the virtual caregiver information based on the feedback.

4. The system of claim 1, wherein the at least one processor is further directed to cause the system to perform operations including:
determining state information of the object, the state information including at least one of an emotional state, a physiological state, or a gaze state of the object; and
determining an instruction with respect to reference information associated with a medical operation performed on the object based on the state information.

5. The system of claim 4, wherein the determining state information of the object includes:
obtaining feature information of the object, the feature information including at least one of an image, a video, an audio, or a physiological signal associated with the object; and
determining the state information of the object based on the feature information using a state determination model.

6. The system of claim 4, wherein
the reference information includes an operational parameter of the medical operation;
the instruction includes a control instruction associated with the operational parameter; and
the at least one processor is further directed to cause the system to perform operations including:
adjusting, based on the control instruction, the operational parameter of the medical operation.

7. The system of claim 1, wherein the at least one processor is further directed to cause the system to perform operations including:
obtaining twin data associated with the object from an operation terminal device;
transmitting the twin data associated with the object to a control terminal device;
obtaining guidance data from the control terminal device, wherein the guidance data is determined based on the twin data associated with the object; and
directing a display device associated with the operation terminal device to present the guidance data.

8. The system of claim 7, wherein the twin data includes at least one of twin data corresponding to the object or twin data corresponding to a medical device corresponding to the operation terminal device.

9. The system of claim 7, wherein the guidance data includes at least one of position guidance data or posture guidance data.

10. The system of claim 7, wherein the at least one processor is further directed to cause the system to perform operations including:
obtaining a control instruction from the control terminal device; and
transmitting the control instruction to the operation terminal device to control a medical device corresponding to the operation terminal device to control the medical operation.

11. A method, comprising:
obtaining a call instruction of virtual caregiver information, wherein the virtual caregiver information includes accompany information associated with a caregiver, the caregiver is related to an object, and the accompany information includes at least one of visual information, auditory information, or tactile information; and
directing a display device to display the virtual caregiver information based on the call instruction.

12. The method of claim 11, wherein before the call instruction of the virtual caregiver information is obtained, the method further comprises:
directing the display device to display a scanning protocol associated with the object based on a call instruction of the scanning protocol.

13. The method of claim 11, the method further comprises:
determining state information of the object, the state information including at least one of an emotional state, a physiological state, or a gaze state of the object; and
determining an instruction with respect to the reference information associated with a medical operation performed on the object based on the state information.

14. The method of claim 11, wherein
the reference information includes an operational parameter of the medical operation;
the instruction includes a control instruction associated with the operational parameter; and
the method further comprises:
adjusting, based on the control instruction, the operational parameter of the medical operation.

15. The method of claim 11, further comprising:
receiving a feedback from the object; and
adjusting the virtual caregiver information based on the feedback.

16. The method of claim 11, further comprising:
obtaining twin data associated with the object from an operation terminal device;
transmitting the twin data associated with the object to a control terminal device;
obtaining guidance data from the control terminal device, wherein the guidance data is determined based on the twin data associated with the object; and
directing the display device associated with the operation terminal device to present the guidance data.

17. The method of claim 16, wherein the twin data includes at least one of twin data corresponding to the object or twin data corresponding to a medical device corresponding to the operation terminal device.

18. The method of claim 16, wherein the guidance data includes at least one of position guidance data or posture guidance data.

19. The method of claim 16, further comprising:
obtaining a control instruction from the control terminal device; and
transmitting the control instruction to the operation terminal device to control a medical device corresponding to the operation terminal device to control the medical operation.

20. A system, comprising:
at least one storage medium including a set of instructions; and
at least one processor in communication with the at least one storage medium, wherein when executing the set of instructions, the at least one processor is directed to cause the system to perform operations including:
obtaining twin data associated with an object from an operation terminal device;
transmitting the twin data associated with the object to a control terminal device;
obtaining guidance data from the control terminal device, wherein the guidance data is determined based on the twin data associated with the object; and
directing a display device associated with the operation terminal device to present the guidance data.
